(19) United States
(12) Patent Application Publication
(10) Pub. No.: US 2010/0316678 A1
(43) Pub. Date: Dec. 16, 2010

(54) COMBINATION METHODS AND COMPOSITIONS FOR TREATMENT OF NEUROPATHIC PAIN

(75) Inventor: Colin Stanley Goodchild, Malvern (AU)

Correspondence Address: SEED INTELLECTUAL PROPERTY LAW GROUP PLLC, 701 FIFTH AVE, SUITE 5400, SEATTLE, WA, 98104, US

(73) Assignee: CNSBio Pty Ltd., Abbotsford (AU)

(21) Appl. No.: 12/666,433

(22) PCT Filed: Jun. 26, 2008
(86) PCT No.: PCT/AU08/00929

§ 371 Date: Aug. 5, 2010

Related U.S. Application Data
(60) Provisional application No. 60/946,923, filed on Jun. 28, 2007.

(51) Int. Cl. A61K 9/00; A61K 31/44; A61K 31/485; A61K 31/5377; A61K 31/165; A61K 31/55; A61K 31/16

(52) U.S. Cl. 424/400; 514/353; 514/282; 514/236.2; 514/627; 514/213.01

(57) ABSTRACT

The present invention relates generally to the field of pain management, and in particular, the management of neuropathic pain. The present invention further provides methods and compositions that treat, alleviate, prevent, diminish or otherwise ameliorate the symptoms of neuropathic pain without inducing overt sedation. The present invention also contemplates combination therapy using one or more NK antagonists in combination with one or more compounds which decrease or inhibit neuronal excitation in the treatment of pain in association with the treatment of a particular disease condition or pathology.

### FIELD

The present invention relates generally to the field of pain management, and in particular, the management of neuropathic pain. Compositions and therapeutic protocols also form part of the present invention.

### BACKGROUND

Bibliographical details of references provided in the subject specification are listed at the end of the specification.

Reference to any prior art in this specification is not, and should not be taken as, an acknowledgment or any form of suggestion that this prior art forms part of the common general knowledge in any country.

Neuropathic pain is often reported as having a lancinating or continuous burning character and is frequently associated with the appearance of abnormal sensory signs such as allodynia and hyperalgesia. Alloydnia is defined as pain resulting from a stimulus that does not normally elicit a painful response, and hyperalgesia is characterized by an increased pain response to normally painful stimuli. Some disorders characterized by neuropathic pain include monoradiculopathies, trigeminal neuralgia, postherpetic neuralgia, phantom limb pain, complex regional pain syndromes, back pain and the various peripheral neuropathies. Neuropathic pain may also be associated with diabetes, radio- or chemo-therapy and infections such as HIV. Neuropathic pain may also result as a side effect of drug treatment or abuse.

For clinical purposes, nociceptive pain can be classified as somatic or visceral. Somatic pain results from prolonged activation of nociceptive receptors in somatic tissues such as a bone, joint, muscle or skin. Visceral pain, on the other hand manifests from activation of nociceptive receptors by pathological mechanisms such as mechanical injury, x-ray irradiation and toxic agents.

Neuropathic pain can be characterized by the following clinical features (Teng and Mekhail Pain Practice 3:8-12, 2003, Rajbhandari et al. Pain 83:627-629, 1999, Melzack et al. Ann NY Acad Sci 933:157-174, 2001):
1. There is the presence of an abnormal, unpleasant sensation (dysesthesia) that frequently has a burning or electrical quality with an occasional paroxysmal, brief, shooting, or stabbing quality.
2. Although the onset of most neuropathic pain is within days after the precipitating injury, there is no absolute temporal relationship to the originating neural trauma such that it can begin weeks, months, or even years later.
3. Pain may be felt in a region of sensory deficit.
4. Non-noxious stimuli may be painful (allodynia).
5. Noxious stimuli may produce greater than normal response (hyperalgesia).
6. There may be an increase in the intensity of pain with repeated stimuli and the pain may persist after the removal of stimuli.

There are no analgesic agents specific for one type of pain component over another and neuropathic and nociceptive pains often respond differently to various analgesics.

There is a need to develop efficacious treatments for short-term and long-term treatment of neuropathic pain.

### SUMMARY

Throughout the specification and the claims which follow, unless the context requires otherwise, the word “comprise”, and variations such as “comprises” and “comprising” will be understood to imply the inclusion of a stated integer or step or group of integers or steps but not the exclusion of any other integer or step or group of integers or steps.

Methods and compositions are provided which treat, alleviate, prevent, diminish or otherwise ameliorate the symptoms associated with neuropathic pain in a subject without inducing overt sedation. Reference to “neuropathic pain” includes the pain associated with tissue injury and the resulting neuropathic pain.

In one aspect, a method is contemplated for inducing an analgesic response to neuropathic pain in a mammal comprising administering to the mammal an amount of a neurokinin (NK) antagonist in combination with a neuronal excitation inhibitor. Neuronal excitation inhibitors include compounds which decrease or inhibit neuronal excitation. Such compounds include, but are not limited to, sodium channel blockers, local anaesthetics, modulators of TRPV1 receptors, NMDA-receptor antagonists, calcium channel antagonists, opioids and modulators of CB2 receptors, which combination is effective in reducing the level of or otherwise ameliorating the sensation of pain associated with neuropathic pain processes without inducing overt sedation. Neuronal excitation inhibitors specifically exclude GABA analogs but do not exclude positive modulators of the GABA receptor such as neurosteroids and benzodiazepines.

As used herein, an NK antagonist is defined as any compound which inhibits, decreases or blocks or otherwise impairs the activity of substance P. Such compounds may either act by directly interacting with substance P or selectively interfere with any of the target receptors for substance P, such as the NK1, NK2 or NK3 receptors.

Examples of NK antagonists are provided herein and in one particular embodiment are NK1 antagonists. In other particular embodiments, the NK antagonists are NK2 or NK3 antagonists.

Another aspect also provides a method of inducing an analgesic response in a mammal suffering neuropathic pain without inducing overt sedation by administering to the mammal one or more NK antagonist concurrently, separately or sequentially with respect to one or more neuronal excitation inhibitors. Compounds which decrease or inhibit neuronal excitation function by reducing, decreasing or blocking pain signals being transmitted to the brain are contemplated. Herein, these compounds will be referred to as “neuronal excitation blockers”, “excitation blockers”, “neuronal excitation inhibitor” and “antagonists of neuronal excitation”. Such compounds include, without being limited to flupirtine or a pharmaceutically acceptable salt, derivative, homolog or analog thereof, retigabine, compounds that cause opening of neuronal potassium channels; compounds that cause closure or blockade of sodium channels such as local anaesthetics (eg lignocaine) lamotrogine or mexiletine; neurosteroids; alpha 2 adrenoceptor agonists; non-steroidal anti-inflammatory (NSAIDS); NMDA-receptor antagonists; and calcium channel antagonists. As used herein, the classes of compounds defined as being compounds which decrease or inhibit neuronal excitation specification exclude GABA analogs. The NK antagonist and the neuronal excitation inhibitor are administered in an amount effective to reduce the symptoms of neuropathic pain without inducing overt sedation. Such an effective amount is considered a synergistic effective amount.

In certain embodiments, the methods herein also include the step of selecting a mammal on the basis of the mammal having neuropathic pain.

In one embodiment, a neuronal excitation inhibitor is an opioid, such as but not limited to fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papveretum, alfentanil, buprenorphine and tramadol and pharmaceutically acceptable salts, derivatives, homologs or analogs thereof as well as opioid agonists.

Another embodiment relates to the use of one or more NK antagonists in combination with flupirtine or a pharmaceutically acceptable salt, derivative, homolog or analog thereof in the manufacture of a medicament for inducing an analgesic response in the treatment of neuropathic pain without inducing overt sedation.

A further embodiment relates to the use of one or more NK antagonists and a neuronal excitation inhibitor, such as flupirtine or a pharmaceutically acceptable salt, derivative, homolog or analog thereof, in the manufacture of one or more separate or combined medicaments for inducing analgesia in response to neuropathic pain without inducing overt sedation. In a further embodiment, the NK antagonist is specific for the NK1 receptor and is combined with a neuronal excitation inhibitor such as flupirtine or retigabine.

Yet another embodiment is directed to the use of one or more NK antagonists and one or more sodium channel blockers in the manufacture of a medicament for inducing analgesia in response to neuropathic pain without inducing overt sedation. Examples of sodium channel blockers include lamotrogine and mexiletine or a pharmaceutically acceptable salt, derivative, homolog or analog thereof.

In addition, the NK antagonist may be used in combination with one or more local anaesthetics such as but not limited to lignocaine, bupivacaine, ropivacaine, and procaine tetracaine or a pharmaceutically acceptable salt, derivative, homolog or analog thereof. Such a combination is proposed to induce analgesia in response to neuropathic pain without inducing overt sedation.

Furthermore, the NK antagonist may be used in combination with one or more modulators of TRPV1 receptors, such as but not limited to capsaicin, capsazepine, Nb-VNA, Nv-VNA, SB-705498 and anandamide or a pharmaceutically acceptable salt, derivative, homolog or analog thereof. Such a combination is proposed to induce analgesia in response to neuropathic pain without inducing overt sedation.

Still further, the NK antagonist may be used in combination with one or more modulators of CB2 receptors such as but not limited to SR144528, AM630 and anandamide or a pharmaceutically acceptable salt, derivative, homolog or analog thereof. Such a combination is proposed to induce analgesia in response to neuropathic pain without inducing overt sedation.

Reference to a “neuronal excitation inhibitor” may also include a sodium channel blocker, a local anaesthetic, a modulator of TRPV1 receptor and/or modulator of CB2 receptor. Equally, a sodium channel blocker (including Nav1.7 and Nav 1.8), a local anaesthetic, a modulator of TRPV1 receptor and/or modulator of CB2 receptor may also be a neuronal excitation inhibitor.

In a still further embodiment there is provided a delivery system for inducing analgesia in response to neuropathic pain in a mammal comprising an NK antagonist and a compound selected from a compound which decreases or inhibits neuronal excitation, such as a sodium channel blocker, a local anaesthetic, a modulator of TRPV1 receptor, a calcium channel antagonist and a modulator of CB2 receptor. In one aspect the NK antagonist of choice is selected from one or more of Aprepitant, Lanprepitant, CP-99,994, SDZ NKT 343, Ezlopitant, CP-96345, CP-99994, CP-122721, MK-869, GR 205171. RP 67580, Dapitant, Lanepitant, Noloitanium and/or Sarefutant. The delivery system may, for example, be in the form of a cream or injectable, slow or controlled release injectables, sustained release or slow release formulation, or a tamper proof formulation, or a pharmaceutical formulation or coated onto a stent, catheter or other mechanical device designed for use in a medical procedure.

The compounds herein may be administered, inter alia, orally, transmucosally, rectally including via suppository, subcutaneously, intravenously, intramuscularly, intraperitoneally, intragastrically, intranasally, intrathecally, transdermally or intestinally or injected into a joint. In certain aspects, the compounds are orally or transdermally administered.

In yet another embodiment, the combination therapy is in relation to neuropathic pain associated with neurological conditions. Examples of neurological conditions include but are not limited to neural injury, neurological diseases, severe burns, severe trauma, chronic non-neurological diseases, chronic infections, chronic corticosteroid administration, AIDS, and the like. Neural injuries include acute brain injuries, traumatic brain injuries, closed head injuries, stroke, and the like. Neurological diseases include chronic neurodegenerative diseases such as Alzheimer's disease, Parkinson's disease, Huntington's disease, multiple sclerosis, and the like.

Accordingly, this aspect contemplates a treatment protocol for a neurological condition in a subject, said protocol comprising the steps of administering to said subject an effective amount of an NK antagonist and an amount of a neuronal excitation inhibitor effective to reduce the level of or otherwise ameliorate the sensation of pain. The neurological condition may include any of those listed above. Administration of the NK antagonist may be sequential or simultaneous to the administration of the neuronal excitation inhibitor.

In a further embodiment, combination therapy is in relation to reducing pain during the treatment of or amelioration of symptoms of any one or more of the following diseases which cause neuropathic pain or which have a neuropathic pain component: Abdominal Wall Defect, Abdominal Migraine, Achondrogenesis, Achondrogenesis Type IV, Achondrogenesis Type III, Achondroplasia, Achondroplasia Tarda, Achondroplastic Dwarfism, Acquired Immunodeficiency Syndrome (AIDS), Acute Intermittant Porphyria, Acute Porphyrias, Acute Shoulder Neuritis, Acute Toxic Epidermolysis, Adiposa Dolorosa, Adrenal Neoplasm, Adrenomyeloneuropathy, Adult Dermatomyositis, Amyotrophic Lateral Sclerosis, Amyotrophic Lateral Sclerosis-Polyglucosan Bodies, AN, AN 1, AN 2, Anal Rectal Malformations, Anal Stenosis, Arachnitis, Arachnoiditis Ossificans, Arachnoiditis, Arteritis Giant Cell, Arthritis, Arthritis Urethritica, Ascending Paralysis, Astrocytoma Grade I (Benign), Astrocytoma Grade II (Benign), Athetoid Cerebral Palsy, Barrett Esophagus, Barrett Ulcer, Benign Tumors of the Central Nervous System, Bone Tumor-Epidermoid Cyst-Polyposis, Brachial Neuritis, Brachial Neuritis Syndrome, Brachial Plexus Neuritis, Brachial-Plexus-Neuropathy, Brachiocephalic Ischemia, Brain Tumors, Brain Tumors Benign, Brain Tumors Malignant, Brittle Bone Disease, Bullosa Hereditaria, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-african type, Calcaneal Valgus, Calcaneovalgus, Cavernous Lymphangioma, Cavernous Malformations, Central Form Neurofibromatosis, Cervical Spinal Stenosis, Cervical Vertebral Fusion, Charcot's Disease, Charcot-Marie-Tooth, Charcot-Marie-Tooth Disease, Charcot-Marie-Tooth Disease Variant, Charcot-Marie-Tooth-Roussy-Levy Disease, Childhood Dermatomyositis, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Chronic Adhesive Arachnoiditis, Chronic Idiopathic Polyneuritis (CIP), Chronic Inflammatory Demyelinating Polyneuropathy, Chronic Inflammatory Demyelinating Polyradiculoneuropathy, Cicatricial Pemphigoid, Complex Regional Pain Syndrome, Congenital Cervical Synostosis, Congenital Dysmyelinating Neuropathy, Congenital Hypomyelinating Polyneuropathy, Congenital Hypomyelination Neuropathy, Congenital Hypomyelination, Congenital Hypomyelination (Onion Bulb) Polyneuropathy, Congenital Ichthyosiform Erythroderma, Congenital Tethered Cervical Spinal Cord Syndrome, Cranial Arteritis, Crohn's Disease, Cutaneous Porphyrias, Degenerative Lumbar Spinal Stenosis, Demyelinating Disease, Diabetes Mellitus Diabetes Insulin Dependent, Diabetes Mellitus, Diabetes Mellitus Addison's Disease Myxedema, Discoid Lupus, Discoid Lupus Erythematosus, Disseminated Lupus Erythematosus, Disseminated Neurodermatitis, Disseminated Sclerosis, EDS Kyphoscoliotic, EDS Kyphoscoliosis, EDS Mitis Type, EDS Ocular-Scoliotic, Elastosis Dystrophica Syndrome, Encephalofacial Angiomatosis, Encephalotrigeminal Angiomatosis, Enchondromatosis with Multiple Cavernous Hemangiomas, Endemic Polyneuritis, Endometriosis, Eosinophilic Fasciitis, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Familial Lumbar Stenosis, Familial Lymphedema Praecox, Fibromyositis, Fibrositis, Fibrous Ankylosis of Multiple Joints, Fibrous Dysplasia, Fragile X syndrome, Generalized Fibromatosis, Guillain-Barre Syndrome, Hemangiomatosis Chondrodystrophica, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Herpes Zoster, Hodgkin Disease, Hodgkin's Disease, Hodgkin's Lymphoma, Hyperplastic Epidermolysis Bullosa, Hypertrophic Interstitial Neuropathy, Hypertrophic Interstitial Neuritis, Hypertrophic Interstitial Radiculoneuropathy, Hypertrophic Neuropathy of Refsum, Idiopathic Brachial Plexus Neuropathy, Idiopathic Cervical Dystonia, Juvenile (Childhood) Dermatomyositis (JDMS), Juvenile Diabetes, Juvenile Rheumatoid Arthritis, Pes Planus, Leg Ulcer, Lumbar Canal Stenosis, Lumbar Spinal Stenosis, Lumbosacral Spinal Stenosis, Lupus, Lupus, Lupus Erythematosus, Lymphangiomas, Mononeuritis Multiplex, Mononeuritis Peripheral, Mononeuropathy Peripheral, Monostotic Fibrous Dysplasia, Multiple Cartilaginous Enchondroses, Multiple Cartilaginous Exostoses, Multiple Enchondromatosis, Multiple Myeloma, Multiple Neuritis of the Shoulder Girdle, Multiple Osteochondromatosis, Multiple Peripheral Neuritis, Multiple Sclerosis, Musculoskeletal Pain Syndrome, Neuropathic Amyloidosis, Neuropathic Beriberi, Neuropathy of Brachialpelxus Syndrome, Neuropathy Hereditary Sensory Type I, Neuropathy Hereditary Sensory Type II, Nieman Pick disease Type A (acute neuronopathic form), Nieman Pick disease Type B, Nieman Pick Disease Type C (chronic neuronopathic form), Non-Scarring Epidermolysis Bullosa, Ochronotic Arthritis, Ocular Herpes, Onion-Bulb Neuropathy, Osteogenesis Imperfect, Osteogenesis Imperfecta, Osteogenesis Imperfecta Congenita, Osteogenesis Imperfecta Tarda, Peripheral Neuritis, Peripheral Neuropathy, Perthes Disease, Polyarteritis Nodosa, Polymyalgia Rheumatica, Polymyositis and Dermatomyositis, Polyneuritis Peripheral, Polyneuropathy Peripheral, Polyneuropathy and Polyradiculoneuropathy, Polyostotic Fibrous Dysplasia, Polyostotic Sclerosing Histiocytosis, Postmyelographic Arachnoiditis, Primary Progressive Multiple Sclerosis, Psoriasis, Radial Nerve Palsy, Radicular Neuropathy Sensory, Radicular Neuropathy Sensory Recessive, Reflex Sympathetic Dystrophy Syndrome, Relapsing-Remitting Multiple Sclerosis, Sensory Neuropathy Hereditary Type I, Sensory Neuropathy Hereditary Type II, Sensory Neuropathy Hereditary Type I, Sensory Radicular Neuropathy, Sensory Radicular Neuropathy Recessive, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Spina Bifida, Spina Bifida Aperta, Spinal Arachnoiditis, Spinal Arteriovenous Malformation, Spinal Ossifying Arachnoiditis, Spinal Stenosis, Stenosis of the Lumbar Vertebral Canal, Still's Disease, Syringomyelia, Systemic Sclerosis, Talipes Calcaneus, Talipes Equinovarus, Talipes Equinus, Talipes Varus, Talipes Valgus, Tandem Spinal Stenosis, Temporal Arteritis/Giant Cell Arteritis, Temporal Arteritis, Tethered Spinal Cord Syndrome, Tethered Cord Malformation Sequence, Tethered Cord Syndrome, Tethered Cervical Spinal Cord Syndrome, Thalamic Pain Syndrome, Thalamic Hyperesthetic Anesthesia, Trigeminal Neuralgia, Variegate Porphyria and Vertebral Ankylosing Hyperostosis amongst others.

Accordingly, still another embodiment contemplates a treatment protocol for a disease condition in a subject, said protocol comprising the steps of administering to the subject an effective amount of an NK antagonist and an amount of flupirtine or a pharmaceutically acceptable salt, derivative, homolog or analog thereof effective to reduce the level of or otherwise ameliorate the sensation of pain. The disease condition may include any of those listed above. Administration of the NK antagonist may be sequential or simultaneous or independent of the flupirtine. The treatment protocol may also include selecting the subject on the basis of the subject having neuropathic pain.

The NK antagonist(s) may be combined with flupirtine or pharmaceutically acceptable salt, derivative, homolog or analog thereof. The flupirtine may be administered at a dose of between about 0.5 mg/kg and about 20 mg/kg, at intervals of between about 1 hour and about 50 hours, when administered either prior to or following the NK antagonist or in combination with the NK antagonist.

In one embodiment, the mammal is a human. The subject or group of subjects may be selected on the basis of the type of pain experienced. The “type” of pain may also be subjectively determined based on symptoms described by the subject. Hence, a therapeutic protocol is contemplated which comprises selecting a subject on the basis of symptoms of neuropathic pain and administering to the subject an NK antagonist and a neuronal excitation inhibitor wherein the treatment does not cause overt sedation.

A further aspect of the subject invention provides a system for the controlled release of an NK antagonist and one or more neuronal excitation inhibitors, wherein the neuronal excitation inhibitors include sodium channel blockers; local anaesthetics; modulators of TRPV 1 receptors; modulators of CB2 receptors; potassium channel openers; calcium channel antagonists or blockers; opioids; NMDA-receptor antagonists; alpha2 adrenoceptor modulators; wherein the system comprises:

(a) a deposit-core comprising an effective amount of a first active substance and having defined geometric form, and

(b) a support-platform applied to the deposit-core, wherein the support-platform contains a second active substance, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active substance is one of (i) one or more NK antagonists or (ii) one or more neuronal excitation inhibitors selected from a list of compounds including sodium channel blockers; local anaesthetics; modulators of TRPV1 receptors; modulators of CB2 receptors; potassium channel openers; calcium channel antagonists or blockers; opioids; NMDA-receptor antagonists; and alpha2 adrenoceptor modulators. The second active substance may be (i) or (ii) above.

In another aspect, a system is provided for the controlled release for an NK antagonist and one or more neuronal excitation inhibitors selected from a list of compounds including sodium channel blockers; local anaesthetics; modulators of TRPV1 receptors; modulators of CB2 receptors; potassium channel openers; calcium channel antagonists or blockers; opioids; NMDA-receptor antagonists; and alpha2 adrenoceptor modulators, wherein the system comprises:

(a) a deposit-core comprising an effective amount of (1) one or more NK antagonists and (2) one or more neuronal excitation inhibitors, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Another aspect is directed to the use of an NK antagonist and a neuronal excitation inhibitor in the manufacture of a medicament for ameliorating the sensation of pain associated with neuropathic pain processes without inducing overt sedation.

### BRIEF DESCRIPTION OF THE FIGURES

FIG. 1 is a graphical representation of an aprepitant does response curve for reversal of allodynia caused by diabetic neuropathy: comparison with saline controls and GABAPentin.

FIG. 2 is a graphical representation of aprepitant co-administration with Flupirtine dose response curve for reversal of allodynia caused by diabetic neuropathy: comparison with GABAPentin and saline controls.

FIG. 3 is a graphical representation of flupirtine dose response curves for reversal of heat hyperalgesia caused by diabetic neuropathy: effect of coadministration of aprepitant at 3.12 mg/kg.

FIG. 4 is a graphical representation of the effect of GABAPentin alone, or in combination with aprepitant in the treatment of neuropathic pain.

FIG. 5 is a graphical representation of the effect of aprepitant either alone or in combination with Flupirtine or GABAPentin on the reversal of diabetes induced hyperalgesia. All agents are tested at non-sedating doses.

### DETAILED DESCRIPTION

Unless otherwise indicated, the subject description is not limited to specific formulations of components, manufacturing methods, dosage regimes, or the like, as such may vary. It is also to be understood that the terminology used herein is for the purpose of describing particular embodiments only, and is not intended to be limiting.

The singular forms “a”, “an” and “the” include plural aspects unless the context clearly dictates otherwise. Thus, for example, reference to “a treatment” includes a single treatment or multiple treatments; reference to “an opioid” includes a single opioid, as well as two or more opioids; reference to “the invention” includes a single or multiple aspects of an invention; and the like.

The terms “compound”, “agent”, “active agent”, “chemical agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “agent”, “active agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

Reference to a “compound”, “agent”, “active agent”, “chemical agent” “pharmacologically active agent”, “medicament”, “active” and “drug” includes combinations of two or more actives such as two or more opioids. A “combination” also includes multi-part compositions such as a two-part composition where the agents are provided separately and given or dispensed separately or admixed together prior to dispensation.

For example, a multi-part pharmaceutical pack may have two or more active agents maintained separately.

The terms “effective amount” and “therapeutically effective amount” of an agent as used herein mean a sufficient amount of the agent (e.g. flupirtine and/or an opioid) to provide the desired therapeutic or physiological effect or outcome. Such an effect or outcome includes alleviation of pain or the sensation of pain as well as the inducement of an analgesic effect or at least a reduction in neuropathic pain without inducing overt sedation. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, colouring agents, wetting or emulsifying agents, pH buffering agents, preservatives, and the like.

Similarly, a “pharmacologically acceptable” salt, ester, amide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of the condition being treated, elimination of symptoms and/or underlying cause, prevention of the occurrence of symptoms of the condition and/or their underlying cause and improvement or remediation or amelioration of damage following a condition. In this context a “symptom” includes neuropathic pain.

A “subject” as used herein refers to an animal, including a mammal and in particular a human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, patient, animal, host, subject or recipient. The compounds and methods of the present invention have applications in human medicine, veterinary medicine as well as in general, domestic or wild animal husbandry.

As indicated above, in particular aspects the animals are humans or other primates such as orangutangs, gorillas, marmosets, livestock animals, laboratory test animals, companion animals or captive wild animals, as well as avian species.

Examples of laboratory test animals include mice, rats, rabbits, simian animals, guinea pigs and hamsters. Rabbits, rodent and simian animals provide a convenient test system or animal model. Livestock animals include sheep, cows, pigs, goats, horses and donkeys.

In one embodiment, a method is provided for inducing an analgesic response to neuropathic pain without inducing overt sedation in a mammal. In this context the term “mammal” is intended to encompass both humans and other mammals such as laboratory test animals. This aspect also includes, in one embodiment, the step of selecting a subject having neuropathic pain to be the recipient of treatment. The selection process includes an assessment of symptoms of neuropathic pain or a condition likely to result in neuropathic pain.

Throughout this specification, the term “neuropathic pain” is to be understood to mean pain initiated or caused by a primary lesion or dysfunction within the nervous system. Examples of categories of neuropathic pain that may be treated by the methods of the present invention include monoradiculopathies, trigeminal neuralgia, postherpetic neuralgia, phantom limb pain, complex regional pain syndromes, back pain, neuropathic pain associated with AIDS and infection with the human immunodeficiency virus and the various peripheral neuropathies, including, but not limited to drug-induced and diabetic neuropathies.

In one embodiment, the method herein described induces an analgesic response to neuropathic pain without inducing overt sedation. A subject, in this context, is also referred to as a “patient”, “target” or “recipient”. In this context the terms “analgesia” and “analgesic response” are intended to describe a state of reduced sensibility to pain, which occurs without overt sedation. In related aspects, analgesia occurs without an effect upon the sense of touch. The sensibility to pain may be reduced by at least 30%, at least 50%, at least 70% or at least 85%. In one aspect of the present invention, the sensibility to the neuropathic pain is completely, or substantially completely, removed. To assess the level of reduction of sensibility to pain associated with the analgesia induced by the methods according to the present invention it is possible to conduct tests such as the short form McGill pain questionnaire and/or visual analogue scales for pain intensity and/or verbal rating scales for pain intensity and/or measurement of tactile allodynia using von Frey hairs or similar device. These tests are standard tests within the art and would be well known to the skilled person.

Accordingly, one aspect contemplates a method for inducing an analgesic response to neuropathic pain without inducing overt sedation in a mammal comprising administering to the subject an amount of one or more NK antagonists and a neuronal excitation inhibitor, such as flupirtine or retigabine or a pharmaceutically acceptable salt, derivative, homolog or analog thereof effective to reduce the level of or otherwise ameliorate the sensation of pain.

A method is also provided for inducing an analgesic response to neuropathic pain in a mammal, said method comprising administering to the mammal one or more NK antagonists and one or more neuronal excitation inhibitors, the neuronal excitation inhibitors selected from a list of compounds which decrease or inhibit neuronal excitation including sodium channel blockers; local anaesthetics; modulators of TRPV1 receptors; modulators of CB2 receptors; potassium channel openers; calcium channel antagonists; opioids; NMDA-receptor antagonists; and alpha2 adrenoceptor modulators, in an amount effective to reduce the level of or to otherwise ameliorate the sensation of pain. Neuronal excitation inhibitors do not include GABA analogs.

Another aspect provides a method of inducing analgesia in a mammal suffering neuropathic pain by administering to the mammal one or more of an NK antagonist concurrently, separately or sequentially with respect to a neuronal excitation inhibitor, such as flupirtine or retigabine, or a pharmaceutically acceptable salt, derivative, homolog or analog thereof, in an amount effective to reduce the level of or otherwise ameliorate the sensation of pain associated with neuropathic pain.

Still another aspect contemplates combination therapy in the treatment of a disease associated with neuropathic pain wherein the treatment of the disease, condition or pathology is conducted in association with pain management using one or more NK antagonists and a neuronal excitation inhibitor, such as flupirtine or retigabine or a pharmaceutically acceptable salt, derivative, homolog or analog thereof and optionally an analgesic agent.

Even still another aspect provides a method for inducing an analgesic response to neuropathic pain in a mammal comprising administering to the subject an amount of one or more NK antagonists and a sodium channel blocker such as but not limited to lamotrigine and mexiletine or a pharmaceutically acceptable salt, derivative, homolog or analog thereof to reduce the level of or otherwise ameliorate the sensation of pain.

Another aspect provides a method for inducing an analgesic response to neuropathic pain in a mammal comprising administering to the subject an amount of one or more NK antagonists and a local anaesthetic such as lignocaine, bupivacaine, ropivacaine, and procaine tetracaine or a pharmaceutically acceptable salt, derivative, homolog or analog thereof to reduce the level of or otherwise ameliorate the sensation of pain.

Furthermore, the NK antagonist may be used in combination with one or more modulators of TRPV1 receptors, such as but not limited to capsaicin, capsazepine, Nb-VNA, Nv-VNA, SB-705498 anandamide or a pharmaceutically acceptable salt, derivative, homolog or analog thereof.

Still further, the NK antagonist may be used in combination with one or more modulators of CB2 receptors such as but not limited to SR144528, AM630 and anandamide or a pharmaceutically acceptable salt, derivative, homolog or analog thereof.

In all cases, the analgesic effect is not associated with overt sedation.

By the term “overt sedation” it is intended to convey that the methods (and compositions) of the invention do not result in practically meaningful sedation of the patient or subject being treated, i.e. significant, visible or apparent drowsiness or unconsciousness of the patient being treated. Thus, the treatment methods of the invention do not result in sleepiness or drowsiness in the patient that interfere with, or inhibit, the activities associated with day to day living, such as driving a motor vehicle or operating machinery for human subjects, or feeding and grooming for animal subjects.

The doses of drug combinations have their effects without causing significant cognitive or general impairment of nervous system function (such as attention or wakefulness). Such effects on cognition lead to a change in the measurement that leads to an erroneous conclusion about the drug combination causing analgesia.

The term “NK antagonist” is intended to encompass known and as yet unknown compounds (including pharmaceutically acceptable salts, derivatives, homologs or analogs thereof) which inhibit, decrease or block or otherwise impair the activity of neurokinin 1, neurokinin 2 or substance P. Such compounds can act directly on neurokinin 1, neurokinin 2 or substance P to inhibit its activity or can act on the family of NK receptors such as NK1, NK2 and NK3 receptors. Examples of such agents include achiral pyridine class of neurokinin-1 receptor antagonists; aprepitant; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)—Substance—P (6-11) (sendide); (beta;-Ala(8))—Neurokinin A (4-10); (Tyr(5), D-Trp (6,8,9), Lys-NH(2) (10))—Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b] quinoxaline cyonines as neurokinin antagonists; MEN 10208; DPDTP-octa; GR73632; GR64349; senktide; GR71251; [D-Arg1, D-Pro2, D-Trp 7,9, Leu11]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Trp 7,9 leu11; [Gly6]-NKB [3-10]; [Arg3, D-Ala6]-NKB [3-10]; CP-9634; 3 aminoquinudidine; CP-99994; S18525; 519752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270—pyrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(IH-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperdine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]-and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substituted)-1-azetidinyl]ethyl]-2-piperidines; L760735; L758,298, Cbz-Gly-Leu-Trp-OBzl(CF(3))(2); L733,061; SR144190; SB235375; N—[(R,R)-(E)-1-arylmethyl-3-(2-oxo-azepan-3-yl)carbamoyl]allyl-N-methy-3,5-bis(trifluromethyl)benzamides; 3-[N¹-3,5-bis(trifluromethyl)benzoyl-N-arylmethyl-N¹-methylhydrazino]-N-[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; LY303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; Nurokinin antagonist; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; NK-2 antagonist-Solvat; SLV-332; SLV332, NIK 616; MPV4505; NIK616; MPC 4505; Z501; Z-501; 1 TAK 637; CP 96345; L 659877; CGP 49823; GR 203040; L 732138; S 16474; WIN 51708; ZD 7944; S 18523; CI 1021; PD 154075; :758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363;1 MEN 11149; HSP 117; NIP 530; and AZD 5106. A

s used herein, neuronal excitation inhibitors include, without being limited to, flupirtine or retigabine; compounds which cause opening of neuronal potassium channels, opioids, neurosteroids, NSAIDS, NMDA receptor antagonists and calcium channel antagonists. Compounds which decrease or inhibit neuronal excitement specifically exclude GABA analogs.

Potassium channels openers contemplated for use in the present invention include, without being limited to flupirtine, Retigabine, WAY-133537, ZD6169, Celikalim, NN414, arycyclopropylcarboxylic amides, 3-(pyridinyl-piperazin-1-YL)-phenylethyl amides, cromakalim, pinacidil, P1060, SDZ PC0400, minoxidil, nicorandil, BMS-204352, cromokalim, leveromakalim, lemakalim, diazoxide, charybdotoxin, glyburide and 4-aminopyridine.

Sodium channel blockers include lamotrigine and mexiletine.

Local anaesthetics include lignocaine, bupivacaine, ropivacaine, procaine and tetracaine.

Reference to a “neuronal excitation inhibitor” may also include a sodium channel blocker, a local anaesthetic, a modulator of TRPV1 receptor and/or modulator of CB2 receptor. Equally, a sodium channel blocker, a local anaesthetic, a modulator of TRPV1 receptor and/or modulator of CB2 receptor may also be a neuronal excitation inhibitor.

A modulator of TRPV1 receptor includes but is not limited to capsaicin, capsazepine, Nb-VNA, Nv-VNA, SB-705498 and anandamide or a pharmaceutically acceptable salt, derivative, homolog or analog thereof.

The modulator may be an agonist or an antagonist of the TRPV1 receptor. SB-705498 is an example of an antagonist and capsaicin, capsazepine, Nb-VNA, Nv-VNA anandamide are examples of agonists.

A modulator of CB2 receptor includes but is not limited to SR144528, AM577, AM630 and anandamide or a pharmaceutically acceptable salt, derivative, homolog or analog thereof. The modulator may be an agonist or an antagonist of the CB2 receptor.

As used herein, opioid compounds (opioids) include any compound that is physiologically acceptable in mammalian systems and is a full or at least partial agonist of an opioid receptor. Opioid compounds are well known and include naturally occurring compounds derived from opium such as codeine, morphine and papavarine as well as derivatives of such compounds that generally have structural similarity as well as other structurally unrelated compounds that agonise an opioid receptor present in a mammalian system. Specific examples of opioid compounds contemplated by the present invention include: fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, nalbuprhine papaverine, papaveretum, alfentanil, buprenorphine and tramadol and pharmaceutically acceptable salts, derivatives, homologs or analogs thereof.

Neurosteroids contemplated for use in the present invention include alphadolone and other pregnanediones and salts and derivates thereof (eg alphadolone mono and bi glucuronides) and other neurosteroids that cause antinociception without overt sedation by interaction with spinal cord GABAa receptors.

As used herein, an NMDA receptor antagonist is an agent which blocks or inhibits the activity and/or function of NMDA receptors. Hence, the present invention extends to functional NMDA-receptor antagonists as well as structural NMDA-receptor antagonists. The NMDA receptor is a cell-surface protein complex, widely distributed in the mammalian central nervous system that belongs to the class of ionotropic-glutamate receptors. It is involved in excitatory-synaptic transmission and the regulation of neuronal growth. The structure comprises a ligand-gated/voltage-sensitive ion channel. The NMDA receptor is highly complex and is believed to contain at least five distinct binding (activation) sites: a glycine-binding site, a glutamate-binding site (NMDA-binding site); a PCP-binding site, a polyamine-binding site, and a zinc-binding site. In general, a receptor antagonist is a molecule that blocks or reduces the ability of an agonist to activate the receptor. As used herein, an “NMDA-receptor antagonist” means any compound or composition, known or to be discovered, that when contacted with an NMDA receptor in vivo or in vitro, inhibits the flow of ions through the NMDA-receptor ion channel. A “functional” NMDA antagonist includes agents which raise the threshold for NMDA receptor activation. Activating NMDA receptors increases cell excitability. Any drug that inhibits or decreases neuronal excitation in the CNS can potentially be a “functional” NMDA receptor antagonist because it decreases the excitation caused by NMDA receptor agonists. All such agents may be used in combination with NK antagonists to achieve a desired analgesic effect.

An NMDA-receptor antagonist can contain one or more chiral centers and/or double bonds and, therefore, exist as stereoisomers, such as double-bond isomers (i.e., geometric isomers), enantiomers, or diastereomers. As used herein, the term “NMDA-receptor antagonist” encompass all such enantiomers and stereoisomers, that is, both the stereomerically-pure form (e.g., geometrically pure, enantiomerically pure, or diastereomerically pure) and enantiomeric and stereoisomeric mixtures, e.g., racemates. The term “NMDA-receptor antagonist” further encompasses all pharmaceutically acceptable salts, all complexes (e.g., hydrates, solvates, and clathrates), and all prodrugs of NMDA-receptor antagonist.

NMDA-receptor antagonists suitable for use in the invention can be identified by testing NMDA-receptor antagonists for antinociceptive properties according to standard pain models. See e.g., Sawynok et al. Pain 82:149, 1999; Sawynok et al. Pain 80:45, 1999.

The NMDA-receptor antagonist can be a non-competitive NMDA-receptor antagonist, more preferably, ketamine, even more preferably, ketamine hydrochloride.

As used herein the meaning of the phrase “NMDA-receptor antagonist” encompasses any compound or composition that antagonizes the NMDA receptor by binding at the glycine site. For a review on glycine-site NMDA-receptor antagonists, see Leeson Drug Design for Neuroscience 13:338-381, 1993. Glycine-site NMDA-receptor antagonists can be identified by standard in vitro and in vivo assays. See, for example, the assays described in U.S. Pat. No. 6,251,903 (issued Jun. 26, 2001); U.S. Pat. No. 6,191,165 (issued Feb. 20, 2001; Grimwood et al. Molecular Pharmacology 4:923 1992; Yoneda et al. J Neurochem 62:102, 1994; and Mayer et al. J Neurophysiol 645, 1988, all of which citations are hereby expressly incorporated herein by reference.

Glycine-site NMDA-receptor antagonists include, but are not limited to, glycinamide, threonine, D-serine, felbamate, 5,7-dichlorokynurenic acid, and 3-amino-1-hydroxy-2-pyrrolidone (HA-966), diethylenetriamine, 1,10-diaminodecane, 1,12-diaminododecane, and ifenprodil and those described in U.S. Pat. Nos. 6,251,903; 5,914,403 (issued Jun. 22, 1999); U.S. Pat. No. 5,863,916 (issued Jan. 26, 1999); U.S. Pat. No. 5,783,700 (issued Jul. 21, 1998); and U.S. Pat. No. 5,708,168 (issued Jan. 13, 1998), all of which patents are hereby expressly incorporated herein by reference.

As used herein the meaning of the phrase “NMDA-receptor antagonist” encompasses any compound or composition that antagonizes the NMDA receptor by binding at the glutamate site also referred to herein as “competitive NMDA-receptor antagonists”; see, for example, Olney & Farber Neuropsychopharmacology 13:335, 1995.

Competitive NMDA-receptor antagonists include, but are not limited to, 3-((−)-2-carboxypiperazin-4-ylpropyl-1-phosphate (CPP); 3-(2-carboxypiperzin-4-yl)-prpenyl-1-phosphonate (CPP-ene); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D,L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D,L-alanine; amino-(4-phosphonomethyl-phenyl)-acetic acid (PD 129635); 2-amino-3-(5-chloro-1-phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-3-[2-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2-carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6-phosphonomethyl-decahydro-isoquinoline-3-carboxylic acid (LY 274614); 4-(1H-tetrazol-5-ylmethyl)-piperidine-2-carboxylic acid (LY 233053 and 235723); 6-(1H-Tetrazol-5-ylmethyl)-decahydro-isoquinoline-3-carboxylic acid (LY 233536).

As used herein the meaning of the phrase “NMDA-receptor antagonist” encompasses any compound or composition that antagonizes the NMDA receptor by binding at the PCP (phencyclidine) site, referred to herein as “non-competitive NMDA-receptor antagonists”.

Non-competitive NMDA-receptor antagonists can be identified using routine assays, for example, those described in U.S. Pat. No. 6,251,948 (issued Jun. 26, 2001); U.S. Pat. No. 5,985,586 (issued Nov. 16, 1999), and U.S. Pat. No. 6,025,369 (issued Feb. 15, 2000); Jacobson et al. J Pharmacol Exp Ther 110:243, 1987; and Thurkauf et al. J Med Chem 31:2257, 1988, all of which citations are hereby expressly incorporated herein by reference.

Examples of non-competitive NMDA-receptor antagonists that bind at the PCP site include, but are not limited to, ketamine, phencyclidine, dextromethorphan, dextrorphan, dexoxadrol, dizocilpine (MK-801), remacemide, thienylcyclohexylpiperidine (TCP), N-allylnometazocine (SKF 10,047), cyclazocine, etoxadrol, (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558, tiletamine, kynurenic acid, 7-chloro-kynurenic acid, and memantine; and quinoxalinediones, such as 6-cyano-7-nitroquinoxaline-2,3-dione (CNQX) and 6,7-dinitro-quinoxaline-2,3-dione (DNQX).

As used herein the meaning of “NMDA-receptor antagonist” encompasses compounds that block the NMDA receptor at the polyamine binding site, the zinc-binding site, and other NMDA-receptor antagonists that are either not classified herein according to a particular binding site or that block the NMDA receptor by another mechanism. Examples of NMDA-receptor antagonists that bind at the polyamine site include, but are not limited to, spermine, spermidine, putrescine, and arcaine. Examples of assays useful to identify NMDA-receptor antagonists that act at the zinc or polyamine binding site are disclosed in U.S. Pat. No. 5,834,465 (issued Nov. 10, 1998), hereby expressly incorporated by reference herein.

Other NMDA-receptor antagonists include, but are not limited to, amantadine, eliprodil, lamotrigine, riluzole, aptiganel, flupirtine, retigabine, celfotel, levemopamil, 1-(4-hydroxy-phenyl)-2-(4-phenylsulfanyl-piperidin-1-yl)-propan-1-one; 2-[4-(4-fluoro-benzoyl)-piperidin-1-yl]-1-naphthalen-2-yl-ethanone (E 2001); 3-(1,1-dimethyl-heptyl)-9-hydroxymethyl-6,6-dimethyl-6a,7,8,10a-tetrahydro-6H-benzo[c]chromen-1-ol (HU-211); 1-{4-[1-(4-chloro-phenyl)-1-methyl-ethyl]-2-methoxy-phenyl}-1H-[1,2,4]triazole-3-carboxylic acid amide (CGP 31358); acetic acid 10-hydroxy-7,9,7′,9′-tetramethoxy-3,3′-dimethyl-3,4,3′,4′-tetrahydro-1H,1′H-[5,5′]bi[benzo[g]isochromenyl]-4-yl ester (ES 242-1); 14-hydroxy-11-isopropyl-10-methyl-5-octyl-10,13-diaza-tricyclo[6.6.1. 04,15]pentadeca-1,4,6,8(15)-tetraen-12-one; and 4,5-dioxo-4,5-dihydro-1H-benzo[g]indole-2,7,9-tricarboxylic acid (PQQ) and pharmaceutically acceptable salts thereof.

NSAIDS include, without being limited to, NSAIDS such as acetaminophen (Tylenol, Datril, etc.), aspirin, ibuprofen (Motrin, Advil, Rufen, others), choline magnesium salicylate (Triasate), choline salicylate (Anthropan), diclofenac (voltaren, cataflam), diflunisal (dolobid), etodolac (Iodine), fenoprofen calcium (nalfon), flurobiprofen (ansaid), indomethacin (indocin, indometh, others), ketoprofen (orudis, oruvail), ketorolac tromethamine (toradol), magnesium salicylate (Doan's, magan, mobidin, others), meclofenamate sodium (meclomen), mefenamic acid (relafan), oxaprozin (daypro), piroxicam (feldene), sodium salicylate, sulindac (clinoril), tolmetin (tolectin), meloxicam, nabumetone, naproxen, lornoxicam, nimesulide, indoprofen, remifenzone, salsalate, tiaprofenic acid, flosulide, and the like.

The phrase “pharmaceutically acceptable salt, derivative, homologs or analogs” is intended to convey any pharmaceutically acceptable tautomer, salt, pro-drug, hydrate, solvate, metabolite or other compound which, upon administration to the subject, is capable of providing (directly or indirectly) the compound concerned or a physiologically (e.g. analgesically) active compound, metabolite or residue thereof. An example of a suitable derivative is an ester formed from reaction of an OH or SH group with a suitable carboxylic acid, for example C₁₋₃alkyl-CO₂H, and HO₂C—(CH₂)ₙ—CO₂H (where n is 1-10 such as 1, 2, 3, 4, 5, 6, 7, 8, 9, 10, but preferably 1-4), and CO₂H—CH₂phenyl.

Thus, the active compounds may be in crystalline form, either as the free compounds or as solvates (e.g. hydrates). Methods of solvation are generally known within the art.

The salts of the active compounds herein are pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The term “pro-drug” is used herein in its broadest sense to include those compounds which can be converted in vivo to the compound of interest (e.g. by enzymatic or hydrolytic cleavage). Examples thereof include esters, such as acetates of hydroxy or thio groups, as well as phosphates and sulphonates. Processes for acylating hydroxy or thio groups are known in the art, e.g. by reacting an alcohol (hydroxy group), or thio group, with a carboxylic acid. Other examples of suitable pro-drugs are described in Bundgaard Design of Prodrugs, Elsevier 1985, the disclosure of which is included herein in its entirety by way of reference.

The term “metabolite” includes any compound into which the active agents can be converted in vivo once administered to the subject. Examples of such metabolites are glucuronides, sulphates and hydroxylates.

It will be understood that active agents as described herein may exist in tautomeric forms. The term “tautomer” is used herein in its broadest sense to include compounds capable of existing in a state of equilibrium between two isomeric forms. Such compounds may differ in the bond connecting two atoms or groups and the position of these atoms or groups in the compound. A specific example is keto-enol tautomerism.

The compounds encompassed herein may be electrically neutral or may take the form of polycations, having associated anions for electrical neutrality. Suitable associated anions include sulfate, tartrate, citrate, chloride, nitrate, nitrite, phosphate, perchlorate, halosulfonate or trihalomethylsulfonate.

The active agents may be administered for therapy by any suitable route. It will be understood that the active agents are preferably administered via a route that does not result in overt sedation of the subject. Suitable routes of administration may include oral, rectal, nasal, inhalation of aerosols or particulates, topical (including buccal and sublingual), transdermal, vaginal, intravesical and parenteral (including subcutaneous, intramuscular, intravenous, intrasternal, intra-articular, injections into the joint, intrathecal, epidural and intradermal). Administration of the active agents may be by a route resulting in first presentation of the compound to the stomach of the subject. In one embodiment of the invention, the active agents are administered via an oral route. In another embodiment the active agents are administered by the transdermal route. However it will be appreciated that the route will vary with the condition and age of the subject, the nature of the neuropathic pain being treated, its location within the subject and the judgement of the physician or veterinarian. It will also be understood that individual active agents may be administered by the same or different distinct routes. The individual active agents may be administered separately or together and may be targeted to specific regions of the body.

As used herein, an “effective amount” refers to an amount of active agent that provides the desired analgesic activity when administered according to a suitable dosing regime. The amount of active agent is the amount that provides the desired analgesic activity without causing overt sedation. Dosing may occur at intervals of several minutes, hours, days, weeks or months. Suitable dosage amounts and regimes can be determined by the attending physician or veterinarian. For example, flupirtine or pharmaceutically acceptable salts, derivatives, homologs or analogs thereof, may be administered to a subject at a rate of between about 0.5 to about 20 mg/kg every from about 1 hour to up to about 50 hours, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 hours, such as 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 mg/kg. Particularly useful times are from about 6 hours to about 24 hours, such as 6, 7, 8, 9, 10, 11, 12, 13, 14, 15, 16, 17, 18, 19, 20, 21, 22, 23, 24. Even more particular useful times are between from about 12 to about 24 hours. Such as 12, 13, 14, 15, 16, 17,18, 19, 20, 21, 22, 23 or 24 hours. Dosing of the analgesic agent, such as an opioid, can be determined by the attending physician in accordance with dosing rates in practice. For example, fentanyl can be administered in an amount of about 100 μg whereas morphine may be administered in an amount of 10 mg, also on an hourly basis. The administration amounts may be varied if administration is conducted more or less frequently, such as by continuous infusion, by regular dose every few minutes (e.g. 1, 2, 3 or 4 minutes) or by administration every 5, 10, 20, 30 or 40 minutes (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39 or 40 minutes) or every 1, 2, 3, 4, 5, 6, 7, 8, 9, 10, 11, 12, 13, 14, 15, 16, 17, 18, 19, 20, 21, 22, 23 or 24 hours or up to 50 hours such as, for example, 25, 26, 27, 28, 29, 30, 31, 32, 33, 34, 35, 36, 37, 38, 39, 40, 41, 42, 43, 44, 45, 46, 47, 48, 49 or 50 hours. In many instances administration will be conducted simply on the basis of when the patient requires pain relief.

In one particular embodiment, one or more NK antagonist in combination with the neuronal excitation inhibitor(s) are used to treat neuropathic pain associated with diseases or conditions having a neuropathic pain component.

One embodiment of the present invention contemplates a treatment protocol for neuropathic pain or a condition associated with neuropathic pain in a subject, the protocol comprising the steps of administering to the subject an effective amount of one or more NK antagonist and neuronal excitation inhibitor, without inducing overt sedation. The treatment protocol may also include additional active agents which treat other aspects of a subjects disease. These active agents may be administered sequentially or simultaneously or independently of the neuronal excitation inhibitor and the NK antagonist.

Compositions are also provided comprising one or more NK antagonist or a pharmaceutically acceptable salt, derivative, homolog or analog thereof, with an inhibitor of neuronal excitation together with one or more pharmaceutically acceptable additives and optionally other medicaments. The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions herein suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant, sodium starch glycollate, cross-linked povidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. When reconstituted these can be in the form of aqueous solution, dissolved in water, isotonic saline or a balanced salt solution. Additionally, when reconstituted the product could be a suspension in which the compound(s) is/are dispersed in the liquid medium by combination with liposomes or a lipid emulsion such as soya bean.

Compositions suitable for topical administration to the skin, i.e. transdermal administration, may comprise the active agents dissolved or suspended in any suitable carrier or base and may be in the form of lotions, gels, creams, pastes, ointments and the like. Suitable carriers may include mineral oil, propylene glycol, waxes, polyoxyethylene and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials. The compositions may also be delivered transdermally via a concentration gradient, or an active mechanism such as ionospheres.

The active compounds herein may also be presented as implants, which may comprise a drug bearing polymeric device wherein the polymer is biocompatible and non-toxic. Suitable polymers may include hydrogels, silicones, polyethylenes and biodegradable polymers.

The compounds herein may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms, joint injections, sustained or slow release injectables. For slow release administration, the active ingredients may be suspended as slow release particles or within liposomes, for example.

The compositions herein may be packaged for sale with other active agents or alternatively, other active agents may be formulated with flupirtine or its pharmaceutical salts, derivatives, homologs or analogs thereof and optionally an analgesic agent such as an opioid.

Thus, a further particular aspect provides a system for the controlled release of one or more NK antagonists in combination with a neuronal excitation inhibitor such as flupirtine or retigabine or a pharmaceutically acceptable salt, derivative, homolog or analog thereof, alone or together with another analgesic or active agent, wherein the system comprises:

(a) a deposit-core comprising an effective amount of a first active substance (or the neuronal excitation inhibitor) and having defined geometric form, and

(b) a support-platform applied to the deposit-core, wherein the support-platform contains a second active substance, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active substance is one of (i) one or more NK antagonists or (ii) a neuronal excitation inhibitor. The second active substance may be (i) or (ii) above.

In another aspect, a system is provided for the controlled release for an NK antagonist and a neuronal excitation inhibitor, wherein the system comprises:

(a) a deposit-core comprising an effective amount of (1) one or more NK antagonists and (2) a neuronal excitation inhibitor, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

A further aspect contemplates a system for the controlled release for an NK antagonist and a sodium channel blocker, wherein the system comprises:

(a) a deposit-core comprising an effective amount of (1) one or more NK antagonists and (2) a sodium channel blocker, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Still a further aspect provides a system for the controlled release for an NK antagonist and a local anaesthetic, wherein the system comprises:

(a) a deposit-core comprising an effective amount of (1) one or more NK antagonists and (2) a local anaesthetic, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Even yet a further aspect contemplates a system for the controlled release for an NK antagonist and a modulator of TRPV1 receptor, wherein the system comprises:

(a) a deposit-core comprising an effective amount of (1) one or more NK antagonists and (2) a modulator of TRPV1 receptor, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Another aspect provides a system for the controlled release for an NK antagonist and a modulator of CB2 receptor, wherein the system comprises:

(a) a deposit-core comprising an effective amount of (1) one or more NK antagonists and (2) a modulator of CB2 receptor, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support platform, for example about 15 to 20%. Binder(s), hydrophilic agent(s) and hydrophobic agent(s) typically total up to about 50% by weight of the support platform, for example about 40 to 50%.

The tablet coating may contain one or more water insoluble or poorly soluble hydrophobic excipients. Such excipients may be selected from any of the known hydrophobic cellulosic derivatives and polymers including alkylcellulose, e.g. ethylcellulose, hydroxypropyl cellulose, hydroxypropylmethyl cellulose, carboxymethyl cellulose, and derivatives thereof; polymethacrylic polymers, polyvinyl acetate and cellulose acetate polymers; fatty acids or their esters or salts; long chain fatty alcohols; polyoxyethylene alkyl ethers; polyoxyethylene stearates; sugar esters; lauroyl macrogol-32 glyceryl, stearoyl macrogol-32 glyceryl, and the like. Hydroxypropylmethyl cellulose materials are preferably selected from those low MW and low viscosity materials such as E-Type methocel, and 29-10 types as defined in the USP.

Other agents or excipients that provide hydrophobic quality to coatings may be selected from any waxy substance known for use as tablet excipients. Preferably they have a HLB value of less than 5, and more preferably about 2. Suitable hydrophobic agents include waxy substances such as carnauba wax, paraffin, microcrystalline wax, beeswax, cetyl ester wax and the like; or non-fatty hydrophobic substances such as calcium phosphate salts, e.g. dibasic calcium phosphate.

Preferably the coating contains a calcium phosphate salt, glyceryl behenate, and polyvinyl pyrollidone, or mixtures thereof, and one or more adjuvants, diluents, lubricants or fillers.

Preferred components in the coating are as follows, with generally suitable percentage amounts expressed as percentage weight of the coating.

Polyvinyl pyrollidone (Povidone) is preferably present in amounts of about 1 to 25% by weight or the coating, more particularly 4 to 12%, e.g. 6 to 8%.

Glyceryl behenate is an ester of glycerol and behenic acid (a C22 fatty acid). Glyceryl behenate may be present as its mono-, di-, or tri-ester form, or a mixture thereof. Preferably it has an HLB value of less than 5, more preferably approximately 2. It may be present in amounts of about 5 to 85% by weight of the coating, more particularly from 10 to 70% by weight, and in certain preferred embodiments from 30 to 50%.

Calcium phosphate salt may be the dibasic calcium phosphate dihydrate and may be present in an amount of about 10 to 90% by weight of the coating, preferably 20 to 80%, e.g. 40 to 75%.

The coating may contain other common tablet excipients such as lubricants, colourants, binders, diluents, glidants and taste-masking agents or flavourants.

Examples of excipients include colourants such a ferric oxide, e.g. yellow ferric oxide; lubricants such as magnesium stearate; and glidants such as silicon dioxide, e.g. colloidal silicon dioxide. Yellow ferric oxide may be used in amounts of about 0.01 to 0.5% by weight based on the coating; magnesium stearate may be present in amounts of 1 to 20% by weight of the coating, more preferably 2 to 10%, e.g. 0.5 to 1.0%; and colloidal silica may be used in amounts of 0.1 to 20% by weight of the coating, preferably 1 to 10%, more preferably 0.25 to 1.0%.

The core comprises in addition to a drug substance, a disintegrating agent or mixtures of disintegrating agents used in immediate release formulations and well know to persons skilled in the art. The disintegrating agents useful in the exercise of the present invention may be materials that effervesce and or swell in the presence of aqueous media thereby to provide a force necessary to mechanically disrupt the coating material.

Preferably a core contains, in addition to the drug substance, cross-linked polyvinyl pyrollidone and croscarmellose sodium.

The following is a list of preferred core materials. The amounts are expressed in terms of percentage by weight based on the weight of the core.

Cross-linked polyvinyl pyrollidone is described above and is useful as a disintegrating agent, and may be employed in the core in the amounts disclosed in relation to the core.

Croscarmellose sodium is internally cross-linked sodium carboxymethyl cellulose (also known as Ac-Di-Sol) useful as a disintegrating agent.

Disintegrating agents may be used in amounts of 5 to 30% by weight based on the core. However, higher amounts of certain disintegrants can swell to form matrices that may modulate the release of the drug substance. Accordingly, particularly when rapid release is required after the lag time it is preferred that the disintegrants is employed in amounts of up to 10% by weight, e.g. about 5 to 10% by weight.

The core may additionally comprise common tablet excipients such as those described above in relation to the coating material. Suitable excipients include lubricants, diluents and fillers, including but not limited to lactose (for example the mono-hydrate), ferric oxide, magnesium stearates and colloidal silica.

Lactose monohydrate is a disaccharide consisting of one glucose and one galactose moiety. It may act as a filler or diluent in the tablets of the present invention. It may be present in a range of about 10 to 90%, preferably from 20 to 80%, and in certain preferred embodiments from 65 to 70%.

As stated above, one aspect contemplates that core is correctly located within the coating to ensure that a tablet has the appropriate coating thickness.

In this way, lag times will be reliable and reproducible, and intra-subject and inter-subject variance in bioavailability can be avoided. It is advantageous to have a robust in process control to ensure that tablets in a batch contain cores having the appropriate geometry in relation to the coating. Controls can be laborious in that they require an operator to remove random samples from a batch and to cut them open to physically inspect the quality of the core (i.e. whether it is intact, and whether it is correctly located). Furthermore, if a significant number of tablets from the sample fail, a complete batch of tablets may be wasted. Applicant has found that if one adds to the core a strong colourant such as iron oxide, such that the core visibly contrasts with the coating when as strong light is shone on the tablet, it is possible for any faults in the position or integrity of the core to be picked up automatically by a camera appropriately located adjacent a tabletting machine to inspect tablets as they are ejected therefrom.

Still another aspect provides a composition comprising: (a) one or more NK antagonists; and (b) an immediate release neuronal excitation inhibitor.

Further provided is a method for the delivery of the inventive composition to a subject, the method comprising the step of administering the composition to the subject orally, transdermally, or subdermally, wherein the composition comprises components (a) and (b) as defined above.

In one aspect, a tamper-proof narcotic delivery system is created that provides for full delivery of narcotic medication and for analgesic action on legitimate patients while at the same time effectively eliminating the problem of tampering by diversion, adulteration, or pulverization of the medication for abuse by addicts. The composition and method of the invention are of value to those practiced in the medical arts and simultaneously possess no value or utility to individuals seeking to abuse or profit from the abuse of such analgesics.

It should be understood that in addition to the ingredients particularly mentioned above, the compositions of the present invention may include other agents conventional in the art, having regard to the type of composition in question. For example, agents suitable for oral administration may include such further agents as binders, sweetners, thickeners, flavouring agents, disintegrating agents, coating agents, preservatives, lubricants and/or time delay agents.

The formulation may also contain carriers, diluents and excipients. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA.

In another embodiment, the active agents are administered orally, preferably in the form of a tablet, capsule, lozenge or liquid. The administered composition will preferably include a surfactant and/or solubility improver. A suitable solubility improver is water-soluble polyethoxylated caster oil and an example of a suitable surfactant is Cremophor EL. Dose ranges suitable for the NK antagonists are, for example, 100 to 1500 mg orally, every six hours including 100, 200, 300, 400, 500, 600, 700, 800, 900, 1000, 1100, 1200, 1300, 1400, 1500. Suitable dose ranges for morphine are 2.5 to 20 mg every 3 to 6 hours such as 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 and for oxycodone and other opioids 2 to 50 mg every 3 to 12 hours such as 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50.

In one aspect, fentanyl is administered at a rate and concentration of 100 micrograms/hour.

In another aspect, tramadol is administered at a rate of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour.

In a related aspect an NSAID is administered at 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour.

In a further aspect, a neurosteroid is administered at 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour.

The calcium channel antagonists of the present inventions are administered without being limited to, a rate of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 4950, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400 milligrams/hour.

Mechanical devices are provided for introduction to or in a body or body cavity coated with a sustained or slow release formulation of one or more NK antagonists combined with the neuronal excitation inhibitor. Examples of mechanical devices include stents, catheters, artificial limbs, pins, needles, intrathecal implants and the like. Reference to an “intrathecal implant” includes reference to a cylindrical thread or device comprising a semipermeable membrane which permits passage or partial passage of small molecules (such as nutrients and drugs in and cellular metabolic products out). The implant may also contain genetically modified or cultured cells (including stem cells) which secrete out useful cytokines and other metabolites. The implant may be designed to release molecules (or intake cellular by-products) for days, weeks, months or even years.

Stents, for example, typically have a lumen, inner and outer surfaces, and openings extending from the outer surface to the inner surface. The present invention extends to a method for coating a surface of a stent. At least a portion of the stent is placed in contact with a coating solution containing a coating material to be deposited on the surface of the stent. A thread is inserted through the lumen of the stent, and relative motion between the stent and the thread is produced to substantially remove coating material within the openings.

The thread can have a diameter substantially smaller than the diameter of the lumen. The thread can be inserted through the lumen either after or prior to contacting the stent with the coating solution. Relative motion between the stent and the thread can be produced prior to contacting the stent with the coating solution to clean the stent. The thread can be either a filament or a cable with a plurality of wires. The thread can be made of a metallic or polymeric material.

The stent can be dipped into the coating solution or spray coated with the coating solution. The coating material can include a biocompatible polymer, either with or without a pharmaceutically active compound.

In one embodiment, the relative motion is oscillatory motion produced by a vibrating device. The oscillations can be changed (magnitude and/or frequency) to vary thickness of the coating solution on the stent. In another embodiment, the relative motion is produced by a shaker table. Regardless of the type of motion, the relative motion can be produced either after or while the stent is in contact with the coating solution.

The relative motion between the stent and the thread can include initially moving the stent in a horizontal direction substantially parallel to the length of the thread and subsequently moving the stent in a vertical direction substantially perpendicular to the length of the thread. The movement in the horizontal direction can be repeated, with pauses between repetitions. The movement in the vertical direction can also be repeated, with the horizontal and vertical movements alternating.

In order to smooth the relative motion, the thread can be coupled to a damping compensator. The damping compensator connects the thread to a vibrating device. In one embodiment, the damping compensator comprises first and second filaments connected to the thread.

The relative motion can be motion of the stent along the thread. For example, a first end of the thread can be attached to a first stand at a first height and a second end of the thread is attached to a second stand at a second height. The relative motion is produced by a gravity gradient, with the first height differing from the second height. Furthermore, the stent can be moved back and forth between the first and second stands by sequentially increasing or decreasing at least one of the first and second heights. In this way, multiple coatings can be applied to the stent.

The relative motion can also be rotation of the stent relative to the thread. A stream of gas can be passed along at least a portion of the surface of the stent to rotate the stent relative to the thread. The rotation can also occur in conjunction with other relative motion between the stent and the thread.

An implantable medical device is provided having an outer surface covered at least in part by the compositions of the present invention, a conformal coating of a hydrophobic elastomeric material incorporating an amount of active material therein for timed delivery therefrom and means associated with the conformal coating to provide a non-thrombogenic surface after the timed delivery of the active material.

The conformal coating may comprise an amount of finely divided biologically active material in the hydrophobic elastomeric material.

The active agents for use herein may also be presented for use in veterinary compositions. These may be prepared by any suitable means known in the art. Examples of such compositions include those adapted for:

(a) oral administration, e.g. drenches including aqueous and non-aqueous solutions or suspensions, tablets, boluses, powders, granules, pellets for admixture with feedstuffs, pastes for application to the tongue;

(b) parenteral administration, e.g. subcutaneous, intra-articular, intramuscular or intravenous injection as a sterile solution or suspension or through intra-nasal administration;

(c) topical application, e.g. creams, ointments, gels, lotions, etc.

The present invention will now be further described with reference to the following examples, which are intended for the purpose of illustration only and are not intended to limit the generality of the subject invention as hereinbefore described.

### Example 1

### The Use of NK1 Antagonists in the Treatment of Pain

Experiments were performed on male Wistar rats (wt 150-220g). Two series of experiments were performed in an observer blinded fashion with saline (negative) controls and GABAPentin (positive) controls. The investigations were in two stages:
1. a range of doses of aprepitant (an example of a NK antagonist) given alone and in combination with flupirtine (an example of an inhibitor of neuronal excitation) were tested for sedating effects using the open field activity monitor. In this way it was determined which doses of drug and drug combinations could be used to test for analgesic effects without causing sedation;
2. a range of doses of aprepitant given alone and in combination with flupirtine were tested for the ability to reverse the allodynia and hyperalgesia caused by streptozotocin-induced diabetic neuropathy.

### Open Field Activity Monitor

Individual rats (several rats per treatment group) were placed in an open field activity monitor in which the movement of the rat could be monitored remotely by the frequency and number of interruption of infrared beams directed across the box in a grid. The activity in the monitor was measured for periods of 20 minutes in each rat. This was performed in groups of rats that received (1) control injection of saline, (2) GABAPentin 50 mg/kg as the maximum dose of that drug found previously not to be sedating (drug positive control) administered intraperitoneally, or (3) after pharmacological interventions that involve the administration of aprepitant and flupirtine given at a range of doses alone and in combination. If a rat was sedated by a drug or drug combination, the movements recorded were less. Since rats become habituated to the open field monitor, only one experiment was performed on each rat with this test.

The results for these experiments are shown in Tables 1 and 2. It was concluded from these experiments that aprepitant alone caused no sedating effects up to and including the dose of 6.25 mg/kg and dose of aprepitant up to 3.12 mg/kg could be used in combination with flupirtine 10 mg/kg without causing sedation. These were the upper dose limits for subsequent experiments investigating the analgesic effects of these drugs administered together in combinations.

### Streptozotocin-Induced Diabetic Neuropathy

Rats were injected intraperitoneally with streptozotocin (150 mg per kg total dose—Sapphire Bioscience) dissolved in sodium chloride 0.9%. The induction of diabetes was confirmed one week after injection of streptozotocin and weekly thereafter by measurement of tail vein blood glucose levels with Ames Glucofilm test strips and Ames Glucometer blood glucose meter. Only animals with blood glucose levels greater than or equal to 15 mM and maintained continuously for 5 weeks were deemed to be diabetic. This treatment resulted in greater than 80% of all animals diabetic with peripheral neuropathy. The peripheral neuropathy is characterised by allodynia and hyperalgesia. At the end of five weeks these rats were assessed with the methods of measuring hyperalgesia (paw flick—Hargreaves method) and allodynia (withdrawal from von Frey Hair stimulation at normally non noxious intensity) to elucidate the antinociceptive effects of flupirtine and aprepitant given intraperitoneally alone and in combination (with saline and GABApentin for comparison).

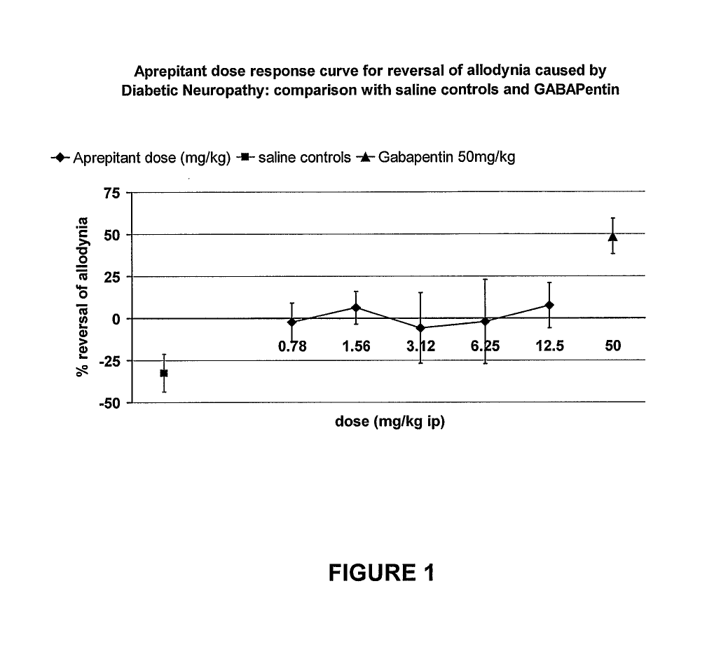

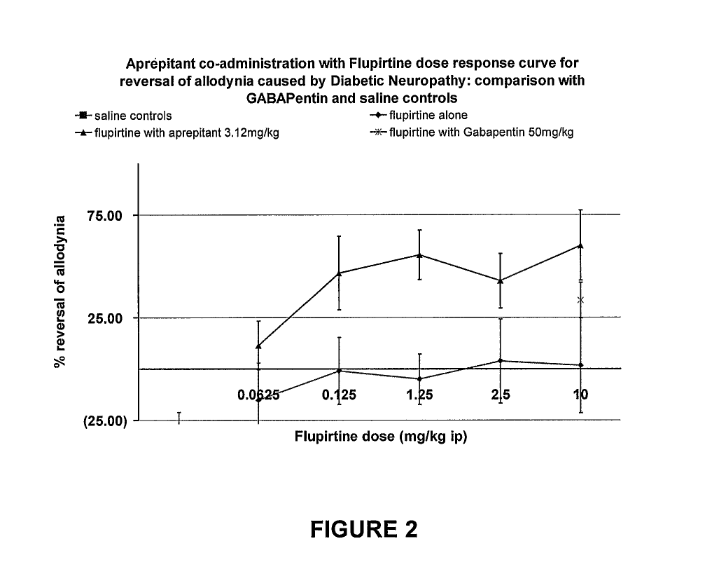

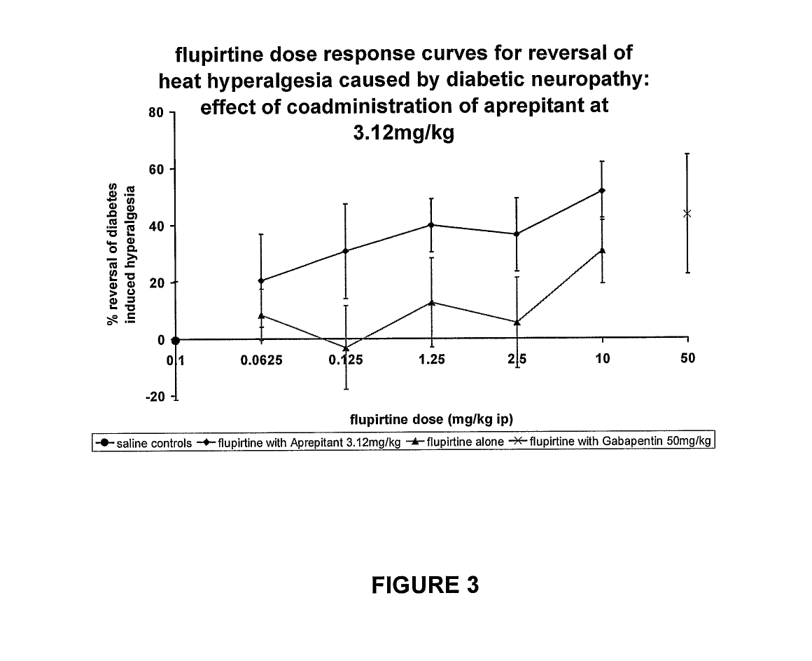

The results of these experiments are shown in FIGS. 1, 2 and 3. It can be seen that with respect to allodynia there is synergy between aprepitant and flupirtine in this model. Neither drug at non sedating doses causes significant antinociception. However, when the two drugs are used in combination, they caused significant analgesia in this model which is greater than that caused by GABApentin. There was similarly an increase in the anti-hyperalgesia response to flupirtine when it was administered in combination with aprepitant.

It can be concluded from these experiments that there is a synergistic interaction between aprepitant, a neurokinin1 receptor antagonist and flupirtine, a KCNQ channel opener in causing anti-nociception and analgesia in models of neuropathic pain at doses that do not cause sedation.

TABLE 1
Results of experiments in normal rats testing for sedation in the open field activity
monitor

| | | Aprep | Aprep | Aprep | Aprep | Aprep |
|---|---|---|---|---|---|---|
| | Saline control | 6.25 mg/kg | 12.5 mg/kg | 25 mg/kg | 50 mg/kg | 100 mg/kg |
| | IP | IP | IP | IP | IP | IP |
| mean | 804.2 | 869.0 | 915.0* | 964.6* | 976.6* | 1058.2 |
| SD | 93.5 | 100.5 | 59.3 | 95.7 | 89.3 | 46.3 |
| n | 20 | 10 | 10 | 10 | 12 | 5 |

*p < 0.01 One way Anova with Dunnett's post hoc test; comparison with saline controls

TABLE 2
Results of experiments in diabetic rats testing for
sedation in the open field activity monitor

| | | | aprepitant |
|---|---|---|---|
| | | | 6.25 mg + |
| | | aprepitant 3.12 mg + | gabapentin |
| | saline controls | flupirtine 10 mg | 50 mg |
| mean | 994.5 | 995.6 | 1003.7 |
| SD | 83.4 | 51.9 | 45.3 |
| n | 12 | 12 | 10 |

Statistical testing: One way ANOVA with Dunnett's post hoc test; p = 0.9357 - no significant sedation caused by aprepitant 3.12 mg/kg administered by intraperitoneal injection in combination with either flupirtine 10 mg/kg or gabapentin 50 mg/kg.

### Example 2

### Use of NK Antagonists in Combination with Modulators of TRPV1 Receptors and CB2 Receptors

It is proposed that the NK antagonists may be used in combination with modulators of TRPV1 receptors and CB2 receptors.

Neuropathic pain is maladaptive and persists following traumatic, metabolic, viral, surgical or drug-induced damage to the sensory nervous system. Examples include postherpetic neuralgia, painful diabetic neuropathy, painful HIV-associated neuropathy and phantom limb pain. There is evidence that nociceptive sensory fibres that survive the traumatic insult or disease process become hyperactive, spontaneously transmitting excessive pain signals to the spinal cord even when no noxious stimuli are present. Nociceptors express the vanilloid receptor 1 (TRPV1 or VR1), a ligand-gated ion channel activated by heat, acidosis or exogenous agonists such as capsaicin. When normal activation occurs, the highly calcium-permeable TRPV1 initiates action potentials that propagate toward the spinal cord. However, when sustained activation of TRPV 1 is induced by exogenous agonists, very high levels of intracellular calcium initiate processes that result in a long-term, yet reversible, cessation of aberrant nociceptor hyperactivity. Capsaicin, one of the exogenous ligands that activate TRPV1, has been used as an analgesic throughout the world. Capsaicin-containing creams, lotions and patches are sold for the treatment of neuralgia, arthritis, and back and muscle pain, as well as for other conditions. Although a promising therapeutic agent, until now capsaicin has not been formulated or delivered in a way that is both clinically effective and tolerable to patients. Recent advances in the understanding of TRPV1 physiology, combined with improved formulations, may help to provide patients with effective, sustained reductions of neuropathic pain without the systemic side effects associated with medications that affect the central nervous system.

Examples of modulators of TRPV1 receptors include capsaicin, capsazepine, Nb-VNA and Nv-VNA as agonists and SB-705498 as antagonist.

Furthermore, the present invention extends to the combination of NK antagonists in combination with cannabinoids which act at CB2 receptors. Such cannabinoids include but are not limited to SR144528, AM577 and AM630 and anandamide; the latter also activates TRPV1 receptors.

### Example 3

### Experiments with Aprepitant and GABAPentin in Streptozotocin-Induced Diabetic Neuropathy

Male Wistar rats (wt 50-70 g) were given streptozotocin 150 mg/kg intraperitoneally. In the following five weeks this treatment caused diabetes mellitus (blood sugar>15 mmol/l) and hyperalgesia. The largest non-sedating doses of GABAPentin (50 mg/kg intraperitoneally) and aprepitant (6.25 mg/kg intraperitoneally) were assessed for possible analgesic effect by measurement of the percentage reversal of the hyperalgesia caused by the diabetes assessed by withdrawal from noxious heat applied to the hind paw.

Experiments were performed in an observer blinded fashion with parallel saline treated controls:

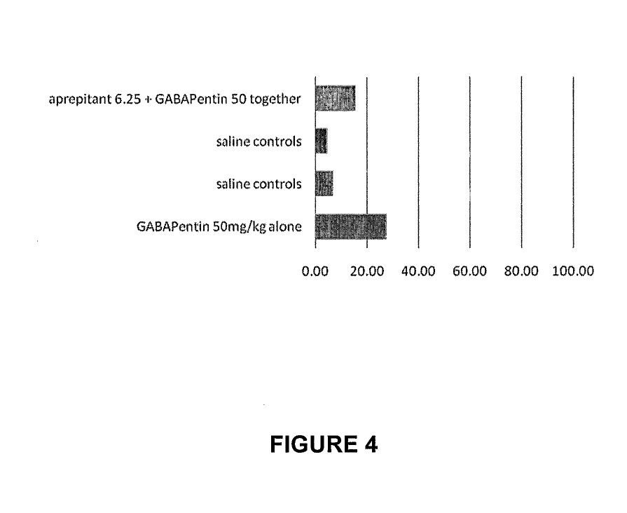

GABAPentin 50 mg/kg intraperitoneally alone (n=17) rats with (n=39) saline controls and aprepitant 6.25 mg/kg given in combination with GABAPentin 50 mg/kg intraperitoneally (n=12) with parallel saline treated controls (n=12). The results of the measurement of antinociception reversal of hyperalgesia are shown in Table 3 and FIG. 4.

TABLE 3

| | GABAPentin | | | aprepitant 6.25 + |
|---|---|---|---|---|
| | 50 mg/kg | saline | | GABAPentin |
| | alone | controls | saline controls | 50 together |
| mean | 27.67 | 6.84 | 4.53 | 15.62 |
| n | 17 | 39 | 16 | 12 |
| SEM | 5.15 | 2.87 | 4.54 | 6.44 |

Statistical analysis of the results shown in FIG. 4 are as follows:
1. GABAPentin 50 alone vs saline **P<0.01
2. GABAPentin 50 alone vs GABAPentin+aprepitant 6.25 mg/kg ns P>0.05
3. saline vs GABAPentin alone+aprepitant ns P>0.05

GABAPentin 50 mg/kg given alone caused statistically significant reversal of hyperalgesia caused by streptozotocin-induced diabetic neuropathy. However, the combination of aprepitant 6.25 mg/kg with this dose of GABAPentin caused no statistically significant antinociception compared with saline and there was no difference compared with GABAPentin alone. Therefore, there appears to be no synergy in causing antinociception between GABAPentin and aprepitant at doses up to the maximal non-sedating doses of each drug.

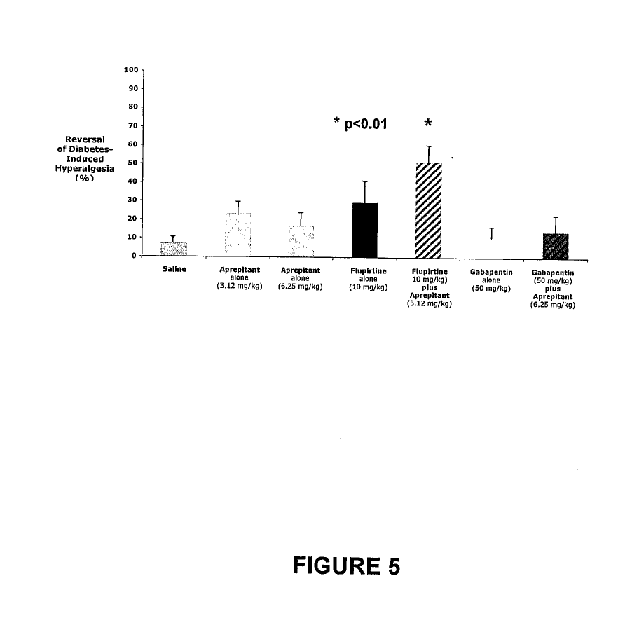

FIG. 5 provides a summary of the data comparing the administration of aprepitant alone, or in combination with either flupirtine or GABAPentin. All agents have been administered at non-sedating doses. As can be seen from FIG. 5, the combination of aprepitant and Flupirtine provided the greatest reversal of diabetes induced hyperalgesia.

Those skilled in the art will appreciate that the invention described herein is susceptible to variations and modifications other than those specifically described. It is to be understood that the invention includes all such variations and modifications. The present invention also includes all of the steps, features, compositions and compounds referred to, or indicated in this specification, individually or collectively, and any and all combinations of any two or more of said steps or features.

### Bibliography

Bundgaard Design of Prodrugs, Elsevier, 1985

Grimwood et al. Molecular Pharmacology 4:923 1992

Jacobson et al. J Pharmacol Exp Ther 110:243, 1987

Leeson Drug Design For Neuroscience 13:338-381, 1993

Mayer et al. J Neurophysiol 645, 1988

Melzack et al. Ann NY Acad Sci 933:157-174, 2001

Olney & Farber Neuropsychopharmacology 13:335, 1995

Rajbhandari et al. Pain 83:627-629, 1999

Remmingtons Pharmaceutical Sciences 18^{th} Edition, Mack Publishing Co., Easton, Pa., USA, 1990

Sawynok et al. Pain 80:45, 1999

Sawynok et al. Pain 82:149, 1999

Teng and Mekhail Pain Practice 3:8-12, 2003

Thurkauf et al. J Med Chem 31:2257, 1988,

U.S. Pat. No. 5,708,168

U.S. Pat. No. 5,783,700

U.S. Pat. No. 5,834,465

U.S. Pat. No. 5,863,916

U.S. Pat. No. 5,985,586

U.S. Pat. No. 6,025,369

U.S. Pat. No. 6,191,165

U.S. Pat. No. 6,251,903

U.S. Pat. No. 6,251,948

Yoneda et al. J Neurochem 62:102, 1994

1. A method for inducing an analgesic response to neuropathic pain without inducing overt sedation in a mammal, said method comprising administering to the mammal identified in step (a) one or more NK antagonists and one or more compounds which decrease or inhibit neuronal excitation in an amount effective to reduce the level of or to otherwise ameliorate the neuropathic pain.

2. The method of claim 1, further comprising selecting the mammal on the basis of the mammal having neuropathic pain.

3. The method of claim 1 wherein the compounds which decrease or inhibit neuronal excitation include sodium channel blockers; local anaesthetics; modulators of TRPV1 receptors; modulators of CB2 receptors; potassium channel openers; calcium channel blockers; opioids; GABA receptor modulators; NMDA-receptor antagonists and alpha2 adrenoceptor modulators.

4. The method according to any one of claim 1 or 2 or 3 wherein the NK antagonist is administered concurrently, separately or sequentially to the compounds which decrease or inhibit neuronal excitation.

5. The method of any one of claims 1 to 4 wherein the NK antagonist is selected from achiral pyridine class of neurokinin-1 receptor antagonists; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)—Substance—P (6-11) (sendide); (beta; -Ala(8))—Neurokinin A (4-10); (Tyr(5), D-Trp (6,8,9), Lys-NH(2) (10))—Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b] quinoxaline cyonines as neurokinin antagonists; MEN 10208; DPDTP-octa; GR73632; GR64349; senktide; GR71251; [D-Argl, D-Pro2, D-Trp 7,9, Leu11]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-ArglD-Trp 7,9 leu11; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270—pyrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(IH-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperdine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-N-and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substiuted)-1-azetidinyl]ethyl]-2-piperidines; L760735; L758,298, Cbz-Gly-Leu-Trp-0Bzl(CF(3))(2); L733,061; SR144190; SB235375; N-[(R,R)-(E)-1-arylmethyl-3-(2-oxo-azepan-3-yl)carbamoyl]allyl-N-methy-3,5-bis(trifluromethyl)benzamides; 3-[N¹-3,5-bis(trifluromethyl)benzoyl-N-arylmethyl-N¹-methylhydrazino]-N-[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; SLV-332; SLV332, NIK 616; MPV4505; NIK616; MPC 4505; Z501; Z-501;1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363;1 MEN 11149; HSP 117; NIP 530; and AZD 5106.

6. The method of any one of claims 1 to 4 wherein the inhibitor of neuronal excitation is selected from flupirtine or a pharmaceutically acceptable salt thereof, retigabine or pharmaceutically acceptable salt thereof, a potassium channel opener, an opioid, an NMDA-receptor antagonist, an NSAID, a neurosteroid and calcium channel antagonist.

7. The method of claim 6 wherein the inhibitor of neuronal excitation is flupirtine.

8. The method of claim 6 wherein the potassium channel opener is selected from WAY-133537, ZD6169, Celikalim, NN414, arycyclopropylcarboxylic amides, 3-(pyridinyl-piperazin-1-YL)-phenylethyl amides, cromakalim, pinacidil, P1060, SDZ PC0400, minoxidil, nicrandil, BMS-204352, cromokalim, leveromakalim, lemakalim, diazoxide, charybdotoxin, glyburide, 4-aminopyridine and BgCl2.

9. The method of claim 6 wherein the opioid is selected from fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papveretum, alfentanil, buprenorphine and tramadol and pharmaceutically acceptable derivates, homologs or analogs thereof.

10. The method of claim 6 where in the NMDA-receptor antagonist is selected from glycinamide, threonine, D-serine, felbamate, 5,7-dichlorokynurenic acid, and 3-amino-1-hydroxy-2-pyrrolidone (HA-966), diethylenetriamine, 1,10-diaminodecane, 1,12-diaminododecane, ifenprodil, 3-((−)-2-carboxypiperazin-4-ylpropyl-1-phosphate (CPP); 3-(2-carboxypiperzin-4-yl)-prpenyl-1-phosphonate (CPP-ene); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D,L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D,L-alanine; amino-(4-phosphonomethyl-phenyl)-acetic acid (PD 129635); 2-amino-3-(5-chloro-1-phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-3-[2-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2-carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6-phosphonomethyl-decahydro-isoquinoline-3-carboxylic acid (LY 274614); 4-(1H-tetrazol-5-ylmethyl)-piperidine-2-carboxylic acid (LY 233053 and 235723); 6-(1H-Tetrazol-5-ylmethyl)-decahydro-isoquinoline-3-carboxylic acid (LY 233536), ketamine, phencyclidine, dextromethorphan, dextrorphan, dexoxadrol, dizocilpine (MK-801), remacemide, thienylcyclohexylpiperidine (TCP), N-allylnometazocine (SKF 10,047), cyclazocine, etoxadrol, (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558, tiletamine, kynurenic acid, 7-chloro-kynurenic acid, and memantine; and quinoxalinediones, such as 6-cyano-7-nitroquinoxaline-2,3-dione (CNQX) and 6,7-dinitro-quinoxaline-2,3-dione (DNQX), amantadine, eliprodil, iamotrigine, riluzole, aptiganel, flupirtine, celfotel, levemopamil, 1-(4-hydroxy-phenyl)-2-(4-phenylsulfanyl-piperidin-1-yl)-propan-1-one; 2-[4-(4-fluoro-benzoyl)-piperidin-1-yl]-1-naphthalen-2-yl-ethanone (E 2001); 3-(1,1-dimethyl-heptyl)-9-hydroxymethyl-6,6-dimethyl-6a,7,8,10a-tetrahydro-6H-benzo[c]chromen-1-ol (HU-211); 1-{4-[1-(4-phenyl)-1-methyl-ethyl]-2-methoxy-phenyl}-1H-[1,2,4]tria zole-3-carboxylic acid amide (CGP 31358); acetic acid 10-hydroxy-7,9,7′,9′-tetramethoxy-3,3′-dimethyl-3,4,3′,4′-tetrahydro-1H,1′H-[5,5′]bi[benzo[g]isochromenyl]-4-yl ester (ES 242-1); 14-hydroxy-11-isopropyl-10-methyl-5-octyl-10,13-diaza-tricyclo[6.6.1.04,15]pentadeca-1,4,6,8(15)-tetraen-12-one; and 4,5-dioxo-4,5-dihydro-1H-benzo[g]indole-2,7,9-tricarboxylic acid (PQQ).

11. The method of claim 6 wherein the neurosteroid is selected from alphadolone and other pregnanediones and salts and derivates thereof (eg alphadolone mono and bi glucuronides) and other neurosteroids that cause antinociception without overt sedation by interaction with spinal cord GABAa receptors.

12. The method of claim 6, wherein the NSAID is selected from acetaminophen (Tylenol, Datril, etc.), aspirin, ibuprofen (Motrin, Advil, Rufen, others), choline magnesium salicylate (Triasate), choline salicylate (Anthropan), diclofenac (voltaren, cataflam), diflunisal (dolobid), etodolac (Iodine), fenoprofen calcium (nalfon), flurobiprofen (ansaid), indomethacin (indocin, indometh, others), ketoprofen (orudis, oruvail), ketorolac tromethamine (toradol), magnesium salicylate (Doan's, magan, mobidin, others), meclofenamate sodium (meclomen), mefenamic acid (relafan), oxaprozin (daypro), piroxicam (feldene), sodium salicylate, sulindac (clinoril), tolmetin (tolectin), meloxicam, nabumetone, naproxen, lornoxicam, nimesulide, indoprofen, remifenzone, salsalate, tiaprofenic acid, flosulide, and the like.

13. The method of claim 6 wherein the sodium channel blocker is selected from lamotrogine and mexiletine and the local anaesthetic is selected from lignocaine, bupivacaine, ropivacaine, procaine, and tetracaine.

14. The method of claim 6 wherein the modulator of TRPV1 receptor is selected from capsaicin, capsazepine, Nb-VNA, Nv-VNA, SB-705498 and anandamide and the modulator of CB2 receptor is selected from SR144528, AM630 and anandamide.

15. The method of claim 7 wherein flupirtine is administered in an amount of about 0.25 mg/kg to about 20 mg/kg of body weight.

16. The method of claim 1 wherein the mammal is human.

17. A delivery system for inducing an analgesic response in a mammal having neuropathic pain said delivery system comprising combined or separate formulations of (1) one or more NK antagonists; (2) one or more compounds selected from a list of compounds which decrease or inhibit neuronal excitation: this list includes: sodium channel blockers; local anaesthetics; modulators of TRPV1 receptors; modulators of CB2 receptors; potassium channel openers; calcium channel blockers; NMDA-receptor antagonists; opioids; GABA receptor modulators; alpha2 adrenoceptor modulators; and (3) optionally one or more further active agents.

18. A delivery system inducing an analgesic response to neuropathic pain without inducing overt sedation in a mammal, said method comprising administering to the mammal one or more NK antagonists and one or more compounds selected from a list of compounds which decrease or inhibit neuronal excitation including sodium channel blockers; local anaesthetics; modulators of TRPV1 receptors; modulators of CB2 receptors; potassium channel openers; calcium channel blockers; opioids; GABA receptor modulators; NMDA-receptor antagonists; alpha2 adrenoceptor modulators, in an amount effective to reduce the level of or to otherwise ameliorate the sensation of pain.

19. The delivery system of claim 18 wherein the NK antagonist is administered concurrently, separately or sequentially to the other compounds.

20. The delivery system of any one of claims 17 to 19 wherein the NK antagonist is selected from achiral pyridine class of neurokinin-1 receptor antagonists; netupitant 21; betctupitant 29; elzlopitant; lanepitant; osanetant; telnetant; GR205171; MK 0517; MK517; MEN 11467; nepadutant; MEN 11420; M274773; [Sar (9), Met (02) (11)]-Substance P; Tyr (6), D-Phe (7), D-His (9)—Substance—P (6-11) (sendide); (beta;-Ala(8))—Neurokinin A (4-10); (Tyr(5), D-Trp (6,8,9), Lys-NH(2) (10))—Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Argl, D-Pro2, D-Trp 7,9, Leu11]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Trp 7,9 leu11; [Gly6]-NKB [3-10]; [Arg3, D-Ala6]-NKB [3-10]; CP-9634; 3 aminoquinudidine; CP-99994; 518525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270—pyrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(IH-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperdine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]-and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substiuted)-1-azetidinyl]ethyl]-2-piperidines; L760735; L758,298, Cbz-Gly-Leu-Trp-0Bzl(CF(3))(2); L733,061; SR144190; SB235375; N-[(R,R)-(E)-1-arylmethyl-3-(2-oxo-azepan-3-yl)carbamoyl]allyl-N-methy-3,5-bis(trifluromethyl)benzamides; 3-[N¹-3,5-bis(trifluromethyl)benzoyl-N-arylmethyl-N¹-methylhydrazino]-N-[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; LY303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; Nurokinin antagonist; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; NK-2 antagonist-Solvat; SLV-332; SLV332, NIK 616; MPV4505; NIK616; MPC 4505; Z501; Z-501;1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363;1 MEN 11149; HSP 117; NIP 530; and AZD 5106.

21. The delivery system of any one of claims 17 to 19 wherein the inhibitor of neuronal excitation is selected from flupirtine or a pharmaceutically acceptable salt, retigabine or a pharmaceutically accepted salt, a potassium channel opener, an opioid, an NMDA-receptor antagonist, a neurosteroid, an NSAID and calcium antagonist.

22. The delivery system of claim 21 wherein the inhibitor of neuronal excitation is flupirtine.

23. The delivery system of claim 21 wherein the potassium channel opener is selected from WAY-133537, ZD6169, Celikalim, NN414, arycyclopropylcarboxylic amides, 3-(pyridinyl-piperazin-1-YL)-phenylethyl amides, cromakalim, pinacidil, P1060, SDZ PC0400, minoxidil, nicrandil, BMS-204352, cromokalim, leveromakalim, lemakalim, diazoxide, charybdotoxin, glyburide, 4-aminopyridine and BgCl2.

24. The delivery system of claim 21 wherein the opioid is selected from the list consisting of fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papveretum, alfentanil, buprenorphine and tramadol and pharmaceutically acceptable derivates, homologs or analogs thereof.

25. The delivery system of claim 21 where in the NMDA-receptor antagonist is selected from glycinamide, threonine, D-serine, felbamate, 5,7-dichlorokynurenic acid, and 3-amino-1-hydroxy-2-pyrrolidone (HA-966), diethylenetriamine, 1,10-diaminodecane, 1,12-diaminododecane, ifenprodil, 3-((−)-2-carboxypiperazin-4-ylpropyl-1-phosphate (CPP); 3-(2-carboxypiperzin-4-yl)-prpenyl-1-phosphonate (CPP-ene); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D,L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D,L-alanine; amino-(4-phosphonomethyl-phenyl)-acetic acid (PD 129635); 2-amino-3-(5-chloro-1-phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-3-[2-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2-carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6-phosphonomethyl-decahydro-isoquinoline-3-carboxylic acid (LY 274614); 4-(1H-tetrazol-5-ylmethyl)-piperidine-2-carboxylic acid (LY 233053 and 235723); 6-(1H-Tetrazol-5-ylmethyl)-decahydro-isoquinoline-3-carboxylic acid (LY 233536), ketamine, phencyclidine, dextromethorphan, dextrorphan, dexoxadrol, dizocilpine (MK-801), remacemide, thienylcyclohexylpiperidine (TCP), N-allylnometazocine (SKF 10,047), cyclazocine, etoxadrol, (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558, tiletamine, kynurenic acid, 7-chloro-kynurenic acid, and memantine; and quinoxalinediones, such as 6-cyano-7-nitroquinoxaline-2,3-dione (CNQX) and 6,7-dinitro-quinoxaline-2,3-dione (DNQX), amantadine, eliprodil, iamotrigine, riluzole, aptiganel, flupirtine, celfotel, levemopamil, 1-(4-hydroxy-phenyl)-2-(4-phenylsulfanyl-piperidin-1-yl)-propan-1-one; 2-[4-(4-fluoro-benzoyl)-piperidin-1-yl]-1-naphthalen-2-yl-ethanone (E 2001); 3-(1,1-dimethyl-heptyl)-9-hydroxymethyl-6,6-dimethyl-6a,7,8,10a-tetrahydro -6H-benzo[c]chromen-1-ol (HU-211); 1-{4-[1-(4-chloro-phenyl)-1-methyl-ethyl]-2-methoxy-phenyl}-1H-[1,2,4]triazole-3-carboxylic acid amide (CGP 31358); acetic acid 10-hydroxy-7,9,7′,9′-tetramethoxy-3,3′-dimethyl-3,4,3′,4′-tetrahydro-1H,1′H-[5,5′]bi[benzo[g]isochromenyl]-4-yl ester (ES 242-1); 14-hydroxy-11-isopropyl-10-methyl-5-octyl-10,13-diaza-tricyclo [6.6.1. 04,15]pentadeca-1,4,6,8(15)-tetraen-12-one; and 4,5-dioxo-4,5-dihydro-1H-benzo[g]indole-2,7,9-tricarboxylic acid (PQQ).

26. The delivery system of claim 21 wherein the neurosteroid is selected from alphadolone and other pregnanediones and salts and derivates thereof (eg alphadolone mono and bi glucuronides) and other neurosteroids that cause antinociception without overt sedation by interaction with spinal cord GABAa receptors.

27. The delivery system of claim 21 wherein the NSAID is selected from acetaminophen (Tylenol, Datril, etc.), aspirin, ibuprofen (Motrin, Advil, Rufen, others), choline magnesium salicylate (Triasate), choline salicylate (Anthropan), diclofenac (voltaren, cataflam), diflunisal (dolobid), etodolac (iodine), fenoprofen calcium (nalfon), flurobiprofen (ansaid), indomethacin (indocin, indometh, others), ketoprofen (orudis, oruvail), ketorolac tromethamine (toradol), magnesium salicylate (Doan's, magan, mobidin, others), meclofenamate sodium (meclomen), mefenamic acid (relafan), oxaprozin (daypro), piroxicam (feldene), sodium salicylate, sulindac (clinoril), tolmetin (tolectin), meloxicam, nabumetone, naproxen, lornoxicam, nimesulide, indoprofen, remifenzone, salsalate, tiaprofenic acid, flosulide, and the like.

28. The delivery system of claim 24 wherein the opioid is morphine or a pharmaceutically acceptable salt thereof.

29. The delivery system of claim 19 wherein the sodium channel blocker is selected from lamotrogine and mexiletine and the local anaesthetic is selected from lignocaine, bupivacine, ropivacaine, procaine and tetracaine.

30. The delivery system of claim 19 wherein the modulator of TRPV1 receptor is selected from capsaicin, capsazepine, Nb-VNA, Nv-VNA, SB-705498 and anandamide and the modulator of CB2 receptor is selected from SR144528, AM630 and anandamide.

31. The delivery system of claim 21 wherein flupirtine is administered in an amount of about 0.25 mg/kg to about 20 mg/kg of body weight.

32. A method of treating neuropathic pain associated with a disease or physiological condition in a mammal, said method comprising:
(a) selecting the mammal on the basis of the mammal having neuropathic pain;
(b) administering to said mammal identified in step (a) an effective amount of one or more NK antagonist and one or more compounds selected from list of compounds which decrease or inhibit neuronal excitation including sodium channel blockers; local anaesthetics; modulators of TRPV1 receptors; modulators of CB2 receptors; potassium channel openers; calcium channel blockers; NMDA-receptor antagonists; opioids; GABA receptor modulators; alpha2 adrenoceptor modulators.

33. A method of treating neuropathic pain associated with a disease or physiological condition of claim 32 wherein the inhibitor of neuronal excitation is flupirtine or a pharmaceutically acceptable salt.

34. A method of treating neuropathic pain associated with a disease or physiological condition in a mammal, said method comprising administering to the mammal one or more NK antagonists and one or more compounds selected from a list of compounds which decrease or inhibit neuronal excitation including sodium channel blockers; local anaesthetics; modulators of TRPV 1 receptors; modulators of CB2 receptors; potassium channel openers; calcium channel blockers; opioids; NMDA-receptor antagonists; GABA receptor modulators; alpha2 adrenoceptor modulators, in an amount effective to reduce the level of or to otherwise ameliorate the sensation of pain.

35. The method of treating neuropathic pain associated with a disease or physiological condition according to claim 32 wherein the NK antagonist is administered concurrently, separately or sequentially to the other compound.

36. The method of claim 32 wherein the NK antagonist is selected from achiral pyridine class of neurokinin-1 receptor antagonists; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)—Substance—P (6-11) (sendide); (beta;-Ala(8))—Neurokinin A (4-10); (Tyr(5), D-Trp (6,8,9), Lys-NH(2) (10))—Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Argl, D-Pro2, D-Trp 7,9, Leu11]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-ArglD-Trp 7,9 leu11; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270—pyrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(IH-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperdine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]-and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substiuted)-1-azetidinyl]ethyl]-2-piperidines; L760735; L758,298, Cbz-Gly-Leu-Trp-0Bzl(CF(3))(2); L733,061; SR144190; SB235375; N-[(R,R)-(E)-1-arylmethyl-3-(2-oxo-azepan-3-yl)carbamoyl]allyl-N-methy-3,5-bis(trifluromethyl)benzamides; 3-[N¹-3,5-bis(trifluromethyl)benzoyl-N-arylmethyl-N¹-methylhydrazino]-N-[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; LY303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; Nurokinin antagonist; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; NK-2 antagonist-Solvat; SLV-332; SLV332, NIK 616; MPV4505; NIK616; MPC 4505; 2501; Z-501;1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363;1 MEN 11149; HSP 117; NIP 530; and AZD 5106.

37. The method of claim 32 or 33 wherein the inhibitor of neuronal excitation is selected from flupirtine or a pharmaceutically acceptable salt, retigabine or a pharmaceutically accepted salt, a potassium channel opener, an opioid, an NMDA antagonist, a functional NMDA antagonist, a neurosteroid and calcium antagonist.

38. The method of claim 37 wherein the inhibitor of neuronal excitation is flupirtine.

39. The method of claim 37 wherein the potassium channel opener is selected from WAY-133537, ZD6169, Celikalim, NN414, arycyclopropylcarboxylic amides, 3-(pyridinyl-piperazin-1-YL)-phenylethyl amides, cromakalim, pinacidil, P1060, SDZ PC0400, minoxidil, nicorandil, BMS-204352, cromokalim, leveromakalim, lemakalim, diazoxide, charybdotoxin, glyburide, 4-aminopyridine and BgCl2.

40. The method of claim 37 wherein the opioid is selected from fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papveretum, alfentanil, buprenorphine and tramadol and pharmaceutically acceptable derivates, homologs or analogs thereof.

41. The method of claim 37 where in the NMDA-receptor antagonist is selected from glycinamide, threonine, D-serine, felbamate, 5,7-dichlorokynurenic acid, and 3-amino-1-hydroxy-2-pyrrolidone (HA-966), diethylenetriamine, 1,10-diaminodecane, 1,12-diaminododecane, ifenprodil, 3-((−)-2-carboxypiperazin-4-ylpropyl-1-phosphate (CPP); 3-(2-carboxypiperzin-4-yl)-prpenyl-1-phosphonate (CPP-ene); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D,L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D,L-alanine; amino-(4-phosphonomethyl-phenyl)-acetic acid (PD 129635); 2-amino-3-(5-chloro-1-phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-342-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2-carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6-phosphonomethyl-decahydro-isoquinoline-3-carboxylic acid (LY 274614); 4-(1H-tetrazol-5-ylmethyl)-piperidine-2-carboxylic acid (LY 233053 and 235723); 6-(1H-Tetrazol-5-ylmethyl)-decahydro-isoquinoline-3-carboxylic acid (LY 233536), ketamine, phencyclidine, dextromethorphan, dextrorphan, dexoxadrol, dizocilpine (MK-801), remacemide, thienylcyclohexylpiperidine (TCP), N-allylnometazocine (SKF 10,047), cyclazocine, etoxadrol, (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558, tiletamine, kynurenic acid, 7-chloro-kynurenic acid, and memantine; and quinoxalinediones, such as 6-cyano-7-nitroquinoxaline-2,3-dione (CNQX) and 6,7-dinitro-quinoxaline-2,3-dione (DNQX), amantadine, eliprodil, iamotrigine, riluzole, aptiganel, flupirtine, celfotel, levemopamil, 1-(4-hydroxy-phenyl)-2-(4-phenylsulfanyl-piperidin-1-yl)-propan-1-one; 2-[4-(4-fluoro-benzoyl)-piperidin-1-yl]-1-naphthalen-2-yl-ethanone (E 2001); 3-(1,1-dimethyl-heptyl)-9-hydroxymethyl-6,6-dimethyl-6a,7,8,10a-tetrahydro-6H-benzo[c]chromen-1-ol (HU-211); 1-{4-[1-(4-chloro-phenyl)-1-methyl-ethyl]-2-methoxy-phenyl-1H-[1,2,4]triazole-3-carboxylic acid amide (CGP 31358); acetic acid 10-hydroxy-7,9,7′,9′-tetramethoxy-3,3′-dimethyl-3,4,3′,4′-tetrahydro-1H,1′H-[5,5′]bi[benzo[g]isochromenyl]-4-yl ester (ES 242-1); 14-hydroxy-11-isopropyl-10-methyl-5-octyl-10,13-diaza-tricyclo[6.6.1. 04,15]pentadeca-1,4,6,8(15)-tetraen-12-one; and 4,5-dioxo-4,5-dihydro-1H-benzo[g]indole-2,7,9-tricarboxylic acid (PQQ).

42. The method of claim 37 wherein the neurosteroid is selected from alphadolone and other pregnanediones and salts and derivates thereof (eg alphadolone mono and bi glucuronides) and other neurosteroids that cause antinociception without overt sedation by interaction with spinal cord GABAa receptors.

43. The method of claim 37 wherein the NSAID is selected from acetaminophen (Tylenol, Datril, etc.), aspirin, ibuprofen (Motrin, Advil, Rufen, others), choline magnesium salicylate (Triasate), choline salicylate (Anthropan), diclofenac (voltaren, cataflam), diflunisal (dolobid), etodolac (iodine), fenoprofen calcium (nalfon), flurobiprofen (ansaid), indomethacin (indocin, indometh, others), ketoprofen (orudis, oruvail), ketorolac tromethamine (toradol), magnesium salicylate (Doan's, magan, mobidin, others), meclofenamate sodium (meclomen), mefenamic acid (relafan), oxaprozin (daypro), piroxicam (feldene), sodium salicylate, sulindac (clinoril), tolmetin (tolectin), meloxicam, nabumetone, naproxen, lornoxicam, nimesulide, indoprofen, remifenzone, salsalate, tiaprofenic acid, flosulide, and the like.

44. The method of claim 40 wherein the opioid is morphine or a pharmaceutically acceptable salt thereof.

45. The method of claim 32 wherein the sodium channel blocker is selected from lamotrogine and mexiletine and the local anaesthetic is selected from lignocaine, bupivacaine, ropivacaine, procaine and tetracaine.

46. The method of claim 32 wherein the modulator of TRPV1 receptor is selected from capsaicin, capsazepine, Nb-VNA, Nv-VNA, SB-705498 and anandamide and the modulator of CB2 receptor is selected from SR144528, AM630 and anandamide.

47. The method of claim 33 wherein flupirtine is administered in an amount of about 0.25 mg/kg to about 20 mg/kg of body weight.

48. The method of treating neuropathic pain associated with a disease or physiological condition according to claim 33 wherein the disease is selected from Abdominal Wall Defect, Abdominal Migraine, Achondrogenesis, Achondrogenesis Type IV, Achondrogenesis Type III, Achondroplasia, Achondroplasia Tarda, Achondroplastic Dwarfism, Acquired Immunodeficiency Syndrome (AIDS), Acute Intermittant Porphyria, Acute Porphyrias, Acute Shoulder Neuritis, Acute Toxic Epidermolysis, Adiposa Dolorosa, Adrenal Neoplasm, Adrenomyeloneuropathy, Adult Dermatomyositis, Amyotrophic Lateral Sclerosis, Amyotrophic Lateral Sclerosis-Polyglucosan Bodies, AN, AN 1, AN 2, Anal Rectal Malformations, Anal Stenosis, Arachnitis, Arachnoiditis Ossificans, Arachnoiditis, Arteritis Giant Cell, Arthritis, Arthritis Urethritica, Ascending Paralysis, Astrocytoma Grade I (Benign), Astrocytoma Grade II (Benign), Athetoid Cerebral Palsy, Barrett Esophagus, Barrett Ulcer, Benign Tumors of the Central Nervous System, Bone Tumor-Epidermoid Cyst-Polyposis, Brachial Neuritis, Brachial Neuritis Syndrome, Brachial Plexus Neuritis, Brachial-Plexus-Neuropathy, Brachiocephalic Ischemia, Brain Tumors, Brain Tumors Benign, Brain Tumors Malignant, Brittle Bone Disease, Bullosa Hereditaria, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-african type, Calcaneal Valgus, Calcaneovalgus, Cavernous Lymphangioma, Cavernous Malformations, Central Form Neurofibromatosis, Cervical Spinal Stenosis, Cervical Vertebral Fusion, Charcot's Disease, Charcot-Marie-Tooth, Charcot-Marie-Tooth Disease, Charcot-Marie-Tooth Disease Variant, Charcot-Marie-Tooth-Roussy-Levy Disease, Childhood Dermatomyositis, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Chronic Adhesive Arachnoiditis, Chronic Idiopathic Polyneuritis (CIP), Chronic Inflammatory Demyelinating Polyneuropathy, Chronic Inflammatory Demyelinating Polyradiculoneuropathy, Cicatricial Pemphigoid, Complex Regional Pain Syndrome, Congenital Cervical Synostosis, Congenital Dysmyelinating Neuropathy, Congenital Hypomyelinating Polyneuropathy, Congenital Hypomyelination Neuropathy, Congenital Hypomyelination, Congenital Hypomyelination (Onion Bulb) Polyneuropathy, Congenital Ichthyosiform Erythroderma, Congenital Tethered Cervical Spinal Cord Syndrome, Cranial Arteritis, Crohn's Disease, Cutaneous Porphyrias, Degenerative Lumbar Spinal Stenosis, Demyelinating Disease, Diabetes Mellitus Diabetes Insulin Dependent, Diabetes Mellitus, Diabetes Mellitus Addison's Disease Myxedema, Discoid Lupus, Discoid Lupus Erythematosus, Disseminated Lupus Erythematosus, Disseminated Neurodermatitis, Disseminated Sclerosis, EDS Kyphoscoliotic, EDS Kyphoscoliosis, EDS Mitis Type, EDS Ocular-Scoliotic, Elastosis Dystrophica Syndrome, Encephalofacial Angiomatosis, Encephalotrigeminal Angiomatosis, Enchondromatosis with Multiple Cavernous Hemangiomas, Endemic Polyneuritis, Endometriosis, Eosinophilic Fasciitis, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Familial Lumbar Stenosis, Familial Lymphedema Praecox, Fibromyositis, Fibrositis, Fibrous Ankylosis of Multiple Joints, Fibrous Dysplasia, Fragile X syndrome, Generalized Fibromatosis, Guillain-Barre Syndrome, Hemangiomatosis Chondrodystrophica, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Herpes Zoster, Hodgkin Disease, Hodgkin's Disease, Hodgkin's Lymphoma, Hyperplastic Epidermolysis Bullosa, Hypertrophic Interstitial Neuropathy, Hypertrophic Interstitial Neuritis, Hypertrophic Interstitial Radiculoneuropathy, Hypertrophic Neuropathy of Refsum, Idiopathic Brachial Plexus Neuropathy, Idiopathic Cervical Dystonia, Juvenile (Childhood) Dermatomyositis (JDMS), Juvenile Diabetes, Juvenile Rheumatoid Arthritis, Pes Planus, Leg Ulcer, Lumbar Canal Stenosis, Lumbar Spinal Stenosis, Lumbosacral Spinal Stenosis, Lupus, Lupus, Lupus Erythematosus, Lymphangiomas, Mononeuritis Multiplex, Mononeuritis Peripheral, Mononeuropathy Peripheral, Monostotic Fibrous Dysplasia, Multiple Cartilaginous Enchondroses, Multiple Cartilaginous Exostoses, Multiple Enchondromatosis, Multiple Myeloma, Multiple Neuritis of the Shoulder Girdle, Multiple Osteochondromatosis, Multiple Peripheral Neuritis, Multiple Sclerosis, Musculoskeletal Pain Syndrome, Neuropathic Amyloidosis, Neuropathic Beriberi, Neuropathy of Brachialpelxus Syndrome, Neuropathy Hereditary Sensory Type I, Neuropathy Hereditary Sensory Type II, Nieman Pick disease Type A (acute neuronopathic form), Nieman Pick disease Type B, Nieman Pick Disease Type C (chronic neuronopathic form), Non-Scarring Epidermolysis Bullosa, Ochronotic Arthritis, Ocular Herpes, Onion-Bulb Neuropathy, Osteogenesis Imperfect, Osteogenesis Imperfecta, Osteogenesis Imperfecta Congenita, Osteogenesis Imperfecta Tarda, Peripheral Neuritis, Peripheral Neuropathy, Perthes Disease, Polyarteritis Nodosa, Polymyalgia Rheumatica, Polymyositis and Dermatomyositis, Polyneuritis Peripheral, Polyneuropathy Peripheral, Polyneuropathy and Polyradiculoneuropathy, Polyostotic Fibrous Dysplasia, Polyostotic Sclerosing Histiocytosis, Postmyelographic Arachnoiditis, Primary Progressive Multiple Sclerosis, Psoriasis, Radial Nerve Palsy, Radicular Neuropathy Sensory, Radicular Neuropathy Sensory Recessive, Reflex Sympathetic Dystrophy Syndrome, Relapsing-Remitting Multiple Sclerosis, Sensory Neuropathy Hereditary Type I, Sensory Neuropathy Hereditary Type II, Sensory Neuropathy Hereditary Type I, Sensory Radicular Neuropathy, Sensory Radicular Neuropathy Recessive, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Spina Bifida, Spina Bifida Aperta, Spinal Arachnoiditis, Spinal Arteriovenous Malformation, Spinal Ossifying Arachnoiditis, Spinal Stenosis, Stenosis of the Lumbar Vertebral Canal, Still's Disease, Syringomyelia, Systemic Sclerosis, Talipes Calcaneus, Talipes Equinovarus, Talipes Equinus, Talipes Varus, Talipes Valgus, Tandem Spinal Stenosis, Temporal Arteritis/Giant Cell Arteritis, Temporal Arteritis, Tethered Spinal Cord Syndrome, Tethered Cord Malformation Sequence, Tethered Cord Syndrome, Tethered Cervical Spinal Cord Syndrome, Thalamic Pain Syndrome, Thalamic Hyperesthetic Anesthesia, Trigeminal Neuralgia, Variegate Porphyria and Vertebral Ankylosing Hyperostosis

49. A system for the controlled release of an NK antagonist and a compound selected from a neuronal excitation inhibitor such as flupirtine or retigabine, a sodium channel blocker such as lamotrogine or mexiletine, a local anaesthetic such as lignocaine, bupivacaine, ropivacaine, procaine or tetracaine, a modulator of TRPV 1 receptor such as capsaicin, capsazepine, Nb-VNA, Nv-VNA, SB-705498 and anandamide and a modulator of CB2 receptor such as SR144528, AM630 and anandamide, or a pharmaceutically acceptable salt, derivative, homolog or analog thereof, wherein the system comprises:
(a) a deposit-core comprising an effective amount of a first active substance and having defined geometric form, and
(b) a support-platform applied to the deposit-core, wherein the support-platform contains a second active substance, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

50. A system for the controlled release for an NK antagonist and a compound selected from a neuronal excitation inhibitor such as flupirtine or retigabine, a sodium channel blocker such as lamotrogine or mexiletine, a local anaesthetic such as lignocaine, bupivacaine, ropivacaine or procaine tetracaine, a modulator of TRPV 1 receptor such as capsaicin, capsazepine, Nb-VNA, Nv-VNA, SB-705498 and anandamide and a modulator of CB2 receptor such as SR144528, AM577, AM630 and anandamide, wherein the system comprises:
(a) a deposit-core comprising an effective amount of (1) one or more NK antagonists and (2) one or more compounds selected from a list of compounds which decrease or inhibit neuronal excitation: this list includes: sodium channel blockers; local anaesthetics; modulators of TRPV1 receptors; modulators of CB2 receptors; potassium channel openers; calcium channel blockers; opioids; GABA receptor modulators; alpha2 adrenoceptor modulators, the deposit-core having a defined geometric form; and
(b) a support platform applied to the deposit-core, the support platform comprising at least one compound selected from the group consisting of:
a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

51. A system for the controlled release of claim 49 or 50 wherein the support platform comprise hydroxypropylmethyl cellulose.

52. A system for the controlled release of claim 49 or 50 wherein the support platform comprises a plasticizer, a binder, a hydrophilic agent and a hydrophobic agent.
